(19) United States
(12) Patent Application Publication
(10) Pub. No.: US 2023/0340065 A1
(43) Pub. Date: Oct. 26, 2023

(54) PRAME SPECIFIC T-CELL RECEPTORS AND USES THEREOF

(71) Applicant: MEDIGENE IMMUNOTHERAPIES GMBH, Planegg-Martinsried (DE)

(72) Inventors: Carina WEHNER, Muenchen (DE); Giulia LONGINOTTI, Muenchen (DE)

(21) Appl. No.: 18/028,025

(22) PCT Filed: Sep. 24, 2021
(86) PCT No.: PCT/EP2021/076324

§ 371 Date: Mar. 23, 2023

(30) Foreign Application Priority Data
Sep. 24, 2020 (EP) 20198096.8

(51) Int. Cl. C07K 14/725

(52) U.S. Cl. CPC C07K 14/7051

(57) ABSTRACT

The present invention relates to a T cell receptor (TCR) capable of binding to a polypeptide comprising the amino acid sequence LYVDSLFFL, or a portion thereof, or its HLA-A bound form. The present invention further relates to nucleic acid molecules encoding said TCR, a vector comprising said nucleic acid molecule, as well as a host cell comprising said nucleic acid molecule or vector. The present invention further relates to methods for obtaining said TCR and to pharmaceutical and diagnostic compositions comprising said TCR, said nucleic acid molecule, vector, and/or host cell. The present invention further relates to such pharmaceutical and diagnostic compositions for use in diagnosing, detecting, preventing, and/or treating cancer. Furthermore, the present invention relates to the use of said TCR, nucleic acid molecule, or said vector, for generating modified lymphocytes.

The present invention relates to a T cell receptor (TCR) capable of binding to a polypeptide comprising the amino acid sequence LYVDSLFFL (SEQ ID NO: 2), or a portion thereof, or its HLA-A bound form. The present invention further relates to nucleic acid molecules encoding said TCR, a vector comprising said nucleic acid molecule, as well as a host cell comprising said nucleic acid molecule or vector. The present invention further relates to methods for obtaining said TCR and to pharmaceutical and diagnostic compositions comprising said TCR, said nucleic acid molecule, vector, and/or host cell. The present invention further relates to such pharmaceutical and diagnostic compositions for use in diagnosing, detecting, preventing, and/or treating cancer. Furthermore, the present invention relates to the use of said TCR, nucleic acid molecule, or said vector, for generating modified lymphocytes.

T lymphocytes (or T cells) which form a part of the cell mediated immune system play a major role in the eradication of pathogens. T cells develop in the thymus and express T cell receptor molecules on their surface that allow the recognition of peptides presented on major histocompatibility complex (MHC) molecules which are expressed on nucleated cells (antigen presentation). Antigens of pathogens, i.e. foreign antigens presented by MHC molecules will elicit a powerful T cell response whereas self-antigens usually do not lead to a T cell response due to a negative selection of self-antigen specific T cells in the thymus during the development of such T cells. The immune system can thus discriminate between nucleated cells presenting foreign- or self-antigens and specifically target and eradicate infected cells via potent cytokine release and cellular cytotoxicity mechanisms of the T cells.

The power of the immune system has been recognized as a promising tool for future cancer therapies. In the last decade, research has begun to exploit the unique properties of T cells by using adoptive cell transfer (ACT), which involves the administration of donor-derived lymphocytes, expanded ex vivo. ACT is an attractive concept for the treatment of cancer because it does not require immune-competence of patients, and the specificity of transferred lymphocytes can be targeted against non-mutated and thus poorly immunogenic tumor antigens that typically fail to effectively trigger autologous T cell responses. Although ACT has been shown to be a promising treatment for various types of cancer, its broad application as clinical treatment has been hampered by the need for custom isolation and characterization of tumor-specific T cells from each patient - a process that can be difficult and time-consuming but also often fails to yield high-avidity T cells (Xue et al., Clin Exp Immunol. 2005 February; 139(2): 167-172; Schmitt et al., Hum Gene Ther. 2009 November; 20(11): 1240-1248).

The genetic transfer of tumor antigen-specific T cell receptors (TCRs) into primary T cells can overcome some of the current limitations of ACT, as it allows for the rapid generation of tumor-reactive T lymphocytes with defined antigen specificity even in immunocompromised patients. However, the identification of suitable T cell clones bearing TCRs that specifically recognize tumor antigens and exhibit the desired anti-tumor effects in vivo is still the topic of ongoing research. Considering that in 2012 about 14.1 million new cases of cancer occurred globally and that cancer currently is the cause of about 14.6% of all human deaths worldwide, novel and efficient treatment options are urgently needed. It is the object of the present invention to comply with the needs set out above.

PRAME is a tumor-associated antigen expressed in a wide variety of tumors, preferably melanoma. Further, PRAME has been described as an independent biomarker for metastasis, such as uveal melanoma (Fiedl et al., Clin Cancer Res 2016 March; 22(5): 1234-1242) and as a prognostic marker for DLBCL (Mitsuhashi et al., Hematology 2014, 1/2014). It is not expressed in normal tissues, except testis. This expression pattern is similar to that of other cancer testis (CT) antigens, such as MAGE, BAGE and GAGE. However, unlike these other CT antigens, this gene is also expressed in acute leukemia. The encoded protein acts as a repressor of retinoic acid receptor, and likely confers a growth advantage to cancer cells via this function. Alternative splicing results in multiple transcript variants. PRAME overexpression in triple negative breast cancer has also been found to promote cancer cell motility through induction of the epithelial-to-mesenchymal transition (AI-Khadairi et al., Journal of Translational Medicine 2019; 17: 9). Deletion of PRAME has been reported in chronic lymphocytic leukemia, however, this is not functionally relevant since the gene is not expressed in B cells, and the deletion is a consequence of a physiological immunoglobulin light chain rearrangement.

Considering that in 2012 about 14.1 million new cases of cancer occurred globally and that cancer currently is the cause of about 14.6% of all human deaths worldwide, novel and efficient treatment options are urgently needed.

Accordingly, the technical problem underlying the present invention was to comply with the objectives set out above. The technical problem has been solved by means and methods as described herein, illustrated in the examples and as defined in the claims.

The present invention relates to a T cell receptor (TCR) capable of binding to
a polypeptide comprising or consisting of an amino acid sequence according to amino acid sequence LYVDSLFFL (SEQ ID NO: 2) wherein not more than 4 amino acids have been substituted, or
to a portion of said polypeptide, or
to the respective HLA-A bound form of said polypeptide or portion thereof,
wherein the TCR comprises:
   (A) a CDR3
      (Aa) of the TCR alpha chain comprising or consisting of an amino acid sequence being at least 80%, 85%, 90%, 95%, 96%, 97%, 98%, or 99% similar or identical (preferably identical), or preferably being 100% similar or identical (preferably identical) to SEQ ID NO: 12, and/or
      (Ab) of the TCR beta chain comprising or consisting of an amino acid sequence being at least 80%, 85%, 90%, 95%, 96%, 97%, 98%, or 99% similar or identical, or preferably being 100% similar or identical (preferably identical) to SEQ ID NO: 14,
      or
   (B) a CDR3
      (Ba) of the TCR alpha chain comprising or consisting of an amino acid sequence being at least 80%, 85%, 90%, 95%, 96%, 97%, 98%, or 99% similar or identical (preferably identical), or preferably being 100% similar or identical (preferably identical) to SEQ ID NO: 40, and/or
      (Bb) of the TCR beta chain comprising or consisting of an amino acid sequence being at least 80%, 85%, 90%, 95%, 96%, 97%, 98%, or 99% similar or identical, or preferably being 100% similar or identical (preferably identical) to SEQ ID NO: 42.

As has surprisingly been found in context with the present invention, a portion of the PRAME peptide, i.e. a polypeptide comprising or consisting of an amino acid sequence according to amino acid sequence LYVDSLFFL (SEQ ID NO: 2; PRAME₃₀₁₋₃₀₉) wherein not more than 4 amino acids have been substituted, is presented by cells via human leukocyte antigen class A (HLA-A) and effectively recognized by a TCR as described and provided herein. Binding of a cell comprising a TCR of the present invention to said polypeptide leads to significant IFN-gamma (IFN-γ) secretion and effective killing of such polypeptide-loaded cells by T-cells transduced with a TCR of the present invention. The polypeptide comprising or consisting of an amino acid sequence according to amino acid sequence LYVDSLFFL (SEQ ID NO: 2; PRAME₃₀₁₋₃₀₉) wherein not more than 4 amino acids have been substituted, as further described and specified herein, is also referred to herein as “PRAME_{L-L}-peptide”.

The term “T cell receptor” or “TCR” as used herein includes in all grammatical forms native TCRs as well as TCR variants, fragments and constructs. The term thus includes heterodimers comprising TCR alpha and beta chains as well as multimers and single chain constructs; optionally comprising further domains and/or moieties.

In accordance with the present invention, in its native form, the TCR exists as a complex of several proteins on the surface of T cells. The T cell receptor is composed of two (separate) protein chains, which are produced from the independent T cell receptor alpha and beta (TCR α and TCR β) genes and are called alpha (α-) and beta (β-) chains. Each chain of the TCR possesses one N-terminal immunoglobulin-like (Ig)-variable (V) domain/region, one Ig-constant-like (C) domain/region, a transmembrane/cell membrane-spanning region anchoring the chain in the plasma membrane, and a short cytoplasmic tail at the C-terminal end.

In accordance with the present invention, antigen specificity is conferred by the variable regions of the alpha and beta chain. Both variable domains of the TCR alpha chain and beta chain comprise three hypervariable or complementarity determining regions (CDR1alpha/beta, CDR2alpha/beta and CDR3alpha/beta) surrounded by framework (FR) regions. CDR3 is the prime determinant of antigen recognition and specificity (i.e. the ability to recognize and interact with a specific antigen), whereas CDR1 and CDR2 mainly interact with the MHC molecule presenting the antigenic peptide.

Native TCRs recognize antigenic peptides bound to (“presented/displayed on”) the major histocompatibility complex (MHC) molecules at the surface of an antigen presenting cell. An antigenic peptide presented on a MHC molecule is also referred to herein as a “peptide:MHC complex” or “peptide:HLA(-A) complex”. There are two different classes of MHC molecules: MHC I and MHC II, which present peptides from different cell compartments. MHC class I molecules are expressed on the surface of all nucleated cells throughout the human body and display peptide or protein fragments from intracellular compartments to cytotoxic T cells. In humans, the MHC is also called the human leukocyte antigen (HLA). There are three major types of MHC class 1: HLA-A, HLA-B and HLA-C. Once a TCR binds to its specific peptide:MHC (e.g., peptide:HLA-A) complex, the T cell is activated and exerts biological effector functions.

In one embodiment of the present invention, the TCRs described and provided in accordance with the present invention specifically bind to their antigenic target, i.e. a polypeptide comprising or consisting of an amino acid sequence according to amino acid sequence LYVDSLFFL (SEQ ID NO: 2) wherein not more than 4 amino acids have been substituted (the PRAME_{L-L}-peptide), or to a portion of said polypeptide, or to the respective HLA-A bound form of said polypeptide or portion thereof. The term “specific(ally) binding” as used herein generally indicates that a TCR binds via its antigen binding site more readily to its intended antigenic target than to a random, unrelated non-target antigen. The specific interaction of the antigen-interaction-site with its specific antigen may result as well in a simple binding of said site to the antigen. Moreover, the specific interaction of the antigen-interaction-site with its specific antigen may alternatively result in the initiation of a signal, e.g. due to the induction of a change of the conformation of the antigen, an oligomerization of the antigen, etc. Typically, in this context and in accordance with the present invention, “specific binding” as used herein means a functional affinity as determined by the half-maximal IFN-γ secretion (EC₅₀) higher than 10⁻⁵M or 10⁻⁶M. Preferably, in context with the present invention, binding is considered specific when binding affinity is about 10⁻¹¹ to 10⁻⁸ M (EC₅₀), preferably of about 10⁻¹¹ to 10⁻⁹ M.

As shown herein, the TCR described and provided in context with the present invention recognize the PRAME_{L-L}-peptide, or a portion thereof, as described and specified herein, particularly when presented on a cell via HLA-A molecules (i.e. in its respective HLA-A bound form). An antigenic peptide is said to be present in its “HLA-A bound form” when it forms a complex with an HLA-A molecule (which may be present on the surface of an antigen presenting cell such as a dendritic cell or a tumor cell, or it may be immobilized by for example coating to a bead or plate). In context with the present invention, such HLA-A molecules may be of any (sub-)allele type and particularly comprise HLA-A molecules encoded by alleles HLA-A*24 or HLA-A*02. As such, the TCR described and provided herein particularly binds to a PRAME_{L-L}-peptide, or a portion thereof, as described and specified herein when presented on a cell via HLA-A*24 or HLA-A*02 molecules, i.e. in its respective HLA-A*24 or HLA-A*02 bound form. In a specific embodiment, the HLA-A*24 is an HLA-A*24:02 encoded molecule, and/or the HLA-A*02 is an HLA-A*02:17 encoded molecule. As such, the TCR described and provided herein particularly binds to a PRAME_{L-L}-peptide, or a portion thereof, as described and specified herein when presented on a cell via HLA-A*24:02 or HLA-A*02:17 molecules, i.e. in its respective HLA-A*24:02 or HLA-A*02:17 bound form. In a preferred specific embodiment, the TCR described and provided herein particularly binds to a PRAME_{L-L}-peptide, or a portion thereof, as described and specified herein when presented on a cell via HLA-A*24:02 molecules, i.e. in its respective HLA-A*24:02 bound form.

In accordance with the present invention, as used herein in context with amino acid sequences, the term “similar” means that a given amino acid sequence comprises identical amino acids or only conservative or highly conservative substitutions compared to the amino acid sequence of the respective SEQ ID NO. As used herein, “conservative” substitutions mean substitutions as listed as “Exemplary Substitutions” in Table I below. “Highly conservative” substitutions as used herein mean substitutions as shown under the heading “Preferred Substitutions” in Table I below.

TABLE I

Amino Acid Substitutions

| | | |
|---|---|---|
| Original | Exemplary Substitutions | Preferred Substitutions |
| Ala (A) | val; leu; ile | Val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| Leu (L) | norleucine; ile; val; met; ala; | ile |
| Lys (K) | arg; gin; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | Phe |
| Val (V) | ile; leu; met; phe; ala; | leu |

The term “amino acid” or “amino acid residue” as used herein typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

The term “position” when used in accordance with the present invention means the position of an amino acid within an amino acid sequence depicted herein. The term “corresponding” in this context also includes that a position is not only determined by the number of the preceding nucleotides/amino acids.

The level of identity between two or more sequences (e.g., nucleic acid sequences or amino acid sequences) can be easily determined by methods known in the art, e.g., by BLAST analysis. Generally, in context with the present invention, if two sequences (e.g., polynucleotide sequences or amino acid sequences) to be compared by, e.g., sequence comparisons differ in identity, then the term “identity” may refer to the shorter sequence and that part of the longer sequence that matches said shorter sequence. Therefore, when the sequences which are compared do not have the same length, the degree of identity may preferably either refer to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that matches the shorter sequence. Furthermore, as used herein, identity levels of nucleic acid sequences or amino acid sequences may refer to the entire length of the respective sequence and is preferably assessed pair-wise, wherein each gap is to be counted as one mismatch. These definitions for sequence comparisons (e.g., establishment of “identity” values) are to be applied for all sequences described and disclosed herein.

Moreover, the term “identity” as used herein means that there is a functional and/or structural equivalence between the corresponding sequences. Nucleic acid/amino acid sequences having the given identity levels to the herein-described particular nucleic acid/amino acid sequences may represent derivatives/variants of these sequences which, preferably, have the same biological function. They may be either naturally occurring variations, for instance sequences from other varieties, species, etc., or mutations, and said mutations may have formed naturally or may have been produced by deliberate mutagenesis. Furthermore, the variations may be synthetically produced sequences. The variants may be naturally occurring variants or synthetically produced variants or variants produced by recombinant DNA techniques.

“Deviations” from sequences (e.g., amino acid or nucleic acid sequences) as used herein may comprise, e.g., deletions, substitutions, additions, insertion and/or recombination. The term “addition” refers to adding a nucleic acid residue/amino acid to the end or beginning of the given sequence, whereas “insertion” refers to inserting a nucleic acid residue/amino acid within a given sequence. The term “deletion” refers to deleting or removal of a nucleic acid residue or amino acid residue in a given sequence. The term “substitution” refers to the replacement of a nucleic acid residue/amino acid residue in a given sequence. Again, these definitions as used here apply, mutatis mutandis, for all sequences provided and described herein unless specified otherwise.

In one embodiment of the present invention, in the polypeptide comprising or consisting of an amino acid sequence according to amino acid sequence LYVDSLFFL (SEQ ID NO: 2) wherein not more than 4 amino acids have been substituted and which the TCR of the present invention (specifically) binds to, positions 2Y and 8F are not substituted. In another embodiment of the present invention, position 6L has only a conservative or preferably highly conservative substitution, or even more preferably, is not substituted. In a specific embodiment of the present invention, said polypeptide (PRAME_{L-L}-peptide) does not have substitutions at positions 2Y, 6L and 8F (vis-à-vis SEQ ID NO: 2). In a more specific embodiment of the present invention, said polypeptide (PRAME_{L-L}-peptide) does not have substitutions at positions 2Y, 5S, 6L, 7F and 8F (vis-à-vis SEQ ID NO: 2).

The term “polypeptide” is equally used herein with the term “protein” or “peptide” unless specifically indicated otherwise. Proteins (including fragments thereof, preferably biologically active fragments, and peptides, usually having less than 30 amino acids) comprise one or more amino acids coupled to each other via a covalent peptide bond (resulting in a chain of amino acids). The term “polypeptide” as used herein describes a group of molecules which typically comprise more than 15 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “polypeptide” and “protein” also refer to naturally modified polypeptides/proteins wherein the modification is effected e.g. by post-translational modifications like glycosylation, acetylation, phosphorylation and the like. Such modifications are well known in the art.

As used herein, in context with a polypeptide, the term “portion” (of a given polypeptide) means a consecutive part of such polypeptide, wherein the N-terminal and/or the C-terminal part of such polypeptide may be deleted. Preferably, as used herein, a “portion” comprises at least 5, more preferably 6 o 7, and most preferably at least 8 consecutive amino acids of said polypeptide. In accordance with the present invention, such “portion” is preferably a “functional portion”, i.e. it is still recognized by a TCR as described and provided herein via (specific) binding and preferably induces IFN-γ secretion by cells comprising said TCR.

In one embodiment of the present invention, binding of the TCR as described and provided herein to the PRAME_{L-L}-peptide as described and further specified herein, or a portion thereof, or its HLA-A bound form as described and specified herein, induces IFN-γ secretion by cells comprising said TCR. In one embodiment, in this context and in accordance with the present invention, the level of IFN-γ secretion of such cell comprising a TCR of the present invention, is at least 3-fold, preferably at least 5-fold, 10-fold or 20-fold higher upon binding to a PRAME_{L-L}-peptide as described an further specified herein (or a portion thereof, or its HLA-A bound form as described and specified herein) compared to a control cell not comprising said TCR, or compared to a cell comprising said TCR binding to an irrelevant peptide (i.e. a peptide which is not a PRAME_{L-L}-peptide as described an further specified herein, or a portion thereof). In context with the present invention, and as also described and exemplified herein, measurement of IFN-gamma can be done by any suitable method known in the art, e.g., ELISA. As an example, for such an assay (e.g., ELISA), the concentration of the PRAME_{L-L}-peptide (and the irrelevant peptide as control) may be about 10⁻⁵M, and the ratio of TCR-comprising cells to targets (PRAME_{L-L}-peptide or portions thereof, alone or in its HLA-A bound form as described and specified herein) may be about 1:2. Cells comprising a TCR as described and provided herein may have received the nucleic acid molecule(s) encoding such TCR either naturally, or preferably via transduction, transfection, or any other suitable methods of stably inserting a nucleic acid molecule into a cell. Suitable cells comprising said TCR are known and the art and are also further described and provided herein as “host cell(s)”. Suitable target cells presenting a PRAME_{L-L}-peptide as described and specified herein, or a portion thereof, are preferably those encoding an HLA-A molecule, to be able to present said PRAME_{L-L}-peptide as described and specified herein, or a portion thereof in its HLA-A bound form via the HLA-A molecule. In this context and in accordance with the present invention, as described herein, specific examples for HLA-A comprise HLA-A*24 (e.g., HLA-A*24:02) and HLA-A*02 (e.g., HLA-A*02:17).

In accordance with the present invention, a TCR (e.g., a native TCR) as described and provided herein is preferably to bind to its antigenic target (i.e PRAME_{L-L}-peptide or portion thereof, or, preferably, its HLA-A bound form, e.g., as presented on HLA-A*24 (e.g., HLA-A*24:02) or HLA-A*02 (e.g., HLA-A*02:17)-encoded molecules by antigen presenting cells, preferably as presented on HLA-A*24:02-encoded molecules by antigen presenting cells) with a high functional avidity. The term “functional avidity” refers to the capability of TCR expressing cells (in particular T cells expressing native TCRs as described herein) to respond in vitro to a given concentration of a ligand, and is thought to correlate with the in vivo effector capacity of TCR expressing cells. By definition, TCR expressing cells with high functional avidity respond in in vitro tests to very low antigen doses, while such cells of lower functional avidity require higher amounts of antigen before they mount an immune response similar to that of high-avidity TCR expressing cells. The functional avidity can be therefore considered as a quantitative determinant of the activation threshold of a TCR expressing cell. It is determined by exposing such cells in vitro to different amounts of cognate antigen. TCR expressing cells with high functional avidity respond to low antigen doses.

For example, a TCR expressing cell will typically be considered to bind with “high” functional avidity to its antigenic target if it secretes about 200 pg/mL or more (e.g. 200 pg/mL or more, 300 pg/mL or more, 400 pg/mL or more, 500 pg/mL or more, 600 pg/mL or more, 700 pg/mL or more, 1000 pg/mL or more, 5000 pg/mL or more, 7000 pg/mL or more, 10000 pg/mL or more, or 20000 pg/mL or more) of interferon gamma (IFN-gamma) upon co-culture with antigen-negative HLA-A (e.g., HLA-A*24 (e.g., HLA-A*24:02) or HLA-A*02 (e.g., HLA-A*02:17) expressing target cells loaded with a low concentration of the PRAME peptide ranging from about 10⁻⁵ to about 10⁻¹¹ M (i.e. about 0.05 ng/mL to about 5 ng/mL, 0.05 ng/mL, 0.1 ng/mL, 0.5 ng/mL, 1 ng/mL, or 5 ng/mL) with the molecular weight of the PRAME peptide having the amino acid sequence according to SEQ ID NO: 2. Hence, the TCR of the present invention is a TCR having a high functional avidity causing a half-maximal relative IFN-γ secretion (EC50 value) of less than 10⁻⁵ M, as measured by an IFN-gamma immunoassay. Preferably, the caused half-maximal relative IFN-gamma secretion (EC50 value) is less than 10⁻⁶ M, as measured by an IFN-gamma immunoassay (cf. FIG. 4 and Example 4).

The cytokine release, such as IFN-gamma secretion, may be measured by any means known in the art and also otherwise exemplified herein, or, e.g., using an in vitro assay in which LCL derived from HLA-A*24:02 or HLA-A*02:17 donors are transfected with ivtRNA or transduced to express the amino acid sequence of, e.g., SEQ ID NO: 2 or irrelevant peptide, respectively, and are incubated with CD8⁺ enriched and/or non-CD8⁺-enriched PBMC expressing the TCR to be investigated or in an in vitro assay using T2 cells externally loaded with either the PRAME peptide according to SEQ ID NO: 2 or the irrelevant peptide and subsequently co-incubated with CD8⁺ enriched and/or non-CD8⁺-enriched PBMC expressing the TCR to be investigated.

In one embodiment of the present invention, the TCR described and provided herein comprising a CDR3 according to (A) further comprises a corresponding CDR1 and/or CDR2 subregion. In one embodiment of the present invention, the TCR described and provided herein comprising a CDR3 according to (A) further comprises
(Aa1) a CDR1 of the TCR alpha chain comprising or consisting of an amino acid sequence being at least 80%, 85%, 90%, 95%, 96%, 97%, 98%, or 99% similar or identical (preferably identical) to the amino acid sequence of SEQ ID NO: 4, and/or a CDR2 of the TCR alpha chain comprising or consisting of an amino acid sequence being at least 80%, 85%, 90%, 95%, 96%, 97%, 98%, or 99% similar or identical (preferably identical) to the amino acid sequence of SEQ ID NO: 8, and/or
(Ab1) a CDR1 of the TCR beta chain comprising or consisting of an amino acid sequence being at least 80%, 85%, 90%, 95%, 96%, 97%, 98%, or 99% similar or identical (preferably identical) to the amino acid sequence of SEQ ID NO: 6, and/or a CDR2 of the TCR beta chain comprising or consisting of an amino acid sequence being at least 80%, 85%, 90%, 95%, 96%, 97%, 98%, or 99% similar or identical (preferably identical) to the amino acid sequence of SEQ ID NO: 10.

In another embodiment of the present invention, the TCR described and provided herein comprising a CDR3 according to (B) further comprises a corresponding CDR1 and/or CDR2 subregion. In one embodiment of the present invention, the TCR described and provided herein comprising a CDR3 according to (B) further comprises
(Ba1) a CDR1 of the TCR alpha chain comprising or consisting of an amino acid sequence being at least 80%, 85%, 90%, 95%, 96%, 97%, 98%, or 99% similar or identical (preferably identical) to the amino acid sequence of SEQ ID NO: 32, and/or a CDR2 of the TCR alpha chain comprising or consisting of an amino acid sequence being at least 80%, 85%, 90%, 95%, 96%, 97%, 98%, or 99% similar or identical (preferably identical) to the amino acid sequence of SEQ ID NO: 36, and/or
(Bb1) a CDR1 of the TCR beta chain comprising or consisting of an amino acid sequence being at least 80%, 85%, 90%, 95%, 96%, 97%, 98%, or 99% similar or identical (preferably identical) to the amino acid sequence of SEQ ID NO: 34, and/or a CDR2 of the TCR beta chain comprising or consisting of an amino acid sequence being at least 80%, 85%, 90%, 95%, 96%, 97%, 98%, or 99% similar or identical (preferably identical) to the amino acid sequence of SEQ ID NO: 38.

In one embodiment of the present invention, the TCR described and provided herein comprising a CDR3 according to (A) comprises
(Aa2) a TCR alpha chain variable region
   comprising or consisting of an amino acid sequence being at least 80%, 85%, 90%, 95%, 96%, 97%, 98%, or 99% similar or identical (preferably identical) to SEQ ID NO: 16, and
   comprising or consisting of an amino acid sequence being at least 80%, 85%, 90%, 95%, 96%, 97%, 98%, or 99% similar or identical (preferably identical) to positions 47 to 51 of SEQ ID NO: 16, and
   comprising or consisting of an amino acid sequence being at least 80%, 85%, 90%, 95%, 96%, 97%, 98%, or 99% similar or identical (preferably identical) to positions 69 to 75 of SEQ ID NO: 16, and
   comprising or consisting of an amino acid sequence being at least 80%, 85%, 90%, 95%, 96%, 97%, 98%, or 99% similar or identical (preferably identical) to positions 109 to 123 of SEQ ID NO: 16,
   and/or
(Ab2) a TCR beta chain variable region comprising or consisting of an amino acid sequence being at least 80%, 85%, 90%, 95%, 96%, 97%, 98%, or 99% similar or identical (preferably identical) to SEQ ID NO: 18, and comprising or consisting of an amino acid sequence being at least 80%, 85%, 90%, 95%, 96%, 97%, 98%, or 99% similar or identical (preferably identical) to positions 46 to 50 of SEQ ID NO: 18, and comprising or consisting of an amino acid sequence being at least 80%, 85%, 90%, 95%, 96%, 97%, 98%, or 99% similar or identical (preferably identical) to positions 68 to 73 of SEQ ID NO: 18, and comprising or consisting of an amino acid sequence being at least 80%, 85%, 90%, 95%, 96%, 97%, 98%, or 99% similar or identical (preferably identical) to positions 110 to 122 of SEQ ID NO: 18.

In another embodiment of the present invention, the TCR described and provided herein comprising a CDR3 according to (B) comprises
(Ba2) a TCR alpha chain variable region
   comprising or consisting of an amino acid sequence being at least 80%, 85%, 90%, 95%, 96%, 97%, 98%, or 99% similar or identical (preferably identical) to SEQ ID NO: 44, and
   comprising or consisting of an amino acid sequence being at least 80%, 85%, 90%, 95%, 96%, 97%, 98%, or 99% similar or identical (preferably identical) to positions 45 to 49 of SEQ ID NO: 44, and
   comprising or consisting of an amino acid sequence being at least 80%, 85%, 90%, 95%, 96%, 97%, 98%, or 99% similar or identical (preferably identical) to positions 67 to 73 of SEQ ID NO: 44, and
   comprising or consisting of an amino acid sequence being at least 80%, 85%, 90%, 95%, 96%, 97%, 98%, or 99% similar or identical (preferably identical) to positions 107 to 121 of SEQ ID NO: 44,
   and/or
(Bb2) a TCR beta chain variable region
   comprising or consisting of the amino acid sequence being at least 80%, 85%, 90%, 95%, 96%, 97%, 98%, or 99% similar or identical (preferably identical) to SEQ ID NO: 46, and
   comprising or consisting of an amino acid sequence being at least 80%, 85%, 90%, 95%, 96%, 97%, 98%, or 99% similar or identical (preferably identical) to positions 44 to 49 of SEQ ID NO: 46, and
   comprising or consisting of an amino acid sequence being at least 80%, 85%, 90%, 95%, 96%, 97%, 98%, or 99% similar or identical (preferably identical) to positions 67 to 71 of SEQ ID NO: 46, and
   comprising or consisting of an amino acid sequence being at least 80%, 85%, 90%, 95%, 96%, 97%, 98%, or 99% similar or identical (preferably identical) to positions 108 to 122 of SEQ ID NO: 46.

In one embodiment of the present invention, the TCR as described and provided herein further comprises (i) a TCR alpha chain constant region, and/or (ii) a TCR beta chain constant region. In one embodiment, the TCR alpha constant region and/or TCR beta chain constant region may be murine (murC), e.g. SEQ ID NO: 24 and SEQ ID NO: 26, respectively, minimally murinized (mmC), e.g. SEQ ID NO: 29 and SEQ ID NO: 30, respectively, or human (huC), e.g. as described herein, such as SEQ ID NO: 28 and SEQ ID NO: 29, respectively. In one embodiment, the TCR alpha constant region and/or TCR beta chain constant region may contain one or more cysteine residues which replace, e.g. a serine or threonine residue such that the TCR alpha constant region can build one or more cysteine bridges with the TCR beta chain constant region, or vice versa, as described, e.g. in Boulter (2003), Protein Engineering 16, 9: 707-711, in particular in Table I on page 708. In one embodiment, in accordance with the present invention, a TCR alpha chain constant region may comprise or consisting of an amino acid sequence being at least 80%, 85%, 90%, 95%, 96%, 97%, 98%, or 99% similar or identical (preferably identical) to SEQ ID NO: 27. In one embodiment, in accordance with the present invention, a TCR beta chain constant region may comprise or consisting of an amino acid sequence being at least 80%, 85%, 90%, 95%, 96%, 97%, 98%, or 99% similar or identical (preferably identical) to SEQ ID NO: 28.

In one embodiment of the present invention, the TCR as described and provided herein comprising a CDR3 according to (A) comprises
(Aa3) a TCR alpha chain
   comprising or consisting of an amino acid sequence being at least 80%, 85%, 90%, 95%, 96%, 97%, 98%, or 99% similar or identical (preferably identical) to SEQ ID NO: 20, and
   comprising or consisting of an amino acid sequence being at least 80%, 85%, 90%, 95%, 96%, 97%, 98%, or 99% similar or identical (preferably identical) to positions 47 to 51 of SEQ ID NO: 20, and
   comprising or consisting of an amino acid sequence being at least 80%, 85%, 90%, 95%, 96%, 97%, 98%, or 99% similar or identical (preferably identical) to positions 69 to 75 of SEQ ID NO: 20, and
   comprising or consisting of an amino acid sequence being at least 80%, 85%, 90%, 95%, 96%, 97%, 98%, or 99% similar or identical (preferably identical) to positions 109 to 123 of SEQ ID NO: 20,
   and/or
(Ab3) a TCR beta chain
   comprising or consisting of an amino acid sequence being at least 80%, 85%, 90%, 95%, 96%, 97%, 98%, or 99% similar or identical (preferably identical) to SEQ ID NO: 22, and
   comprising or consisting of an amino acid sequence being at least 80%, 85%, 90%, 95%, 96%, 97%, 98%, or 99% similar or identical (preferably identical) to positions 46 to 50 of SEQ ID NO: 22, and
   comprising or consisting of an amino acid sequence being at least 80%, 85%, 90%, 95%, 96%, 97%, 98%, or 99% similar or identical (preferably identical) to positions 68 to 73 of SEQ ID NO: 22, and
   comprising or consisting of an amino acid sequence being at least 80%, 85%, 90%, 95%, 96%, 97%, 98%, or 99% similar or identical (preferably identical) to positions 110 to 122 of SEQ ID NO: 22.

In another embodiment of the present invention, the TCR as described and provided herein comprising a CDR3 according to (B) comprises
(Ba3) a TCR alpha chain
   comprising or consisting of an amino acid sequence being at least 80%, 85%, 90%, 95%, 96%, 97%, 98%, or 99% similar or identical (preferably identical) to SEQ ID NO: 48, and
   comprising or consisting of an amino acid sequence being at least 80%, 85%, 90%, 95%, 96%, 97%, 98%, or 99% similar or identical (preferably identical) to positions 45 to 49 of SEQ ID NO: 48, and
   comprising or consisting of an amino acid sequence being at least 80%, 85%, 90%, 95%, 96%, 97%, 98%, or 99% similar or identical (preferably identical) to positions 67 to 73 of SEQ ID NO: 48, and
   comprising or consisting of an amino acid sequence being at least 80%, 85%, 90%, 95%, 96%, 97%, 98%, or 99% similar or identical (preferably identical) to positions 107 to 121 of SEQ ID NO: 48,
   and/or
(Bb3) a TCR beta chain
   comprising or consisting of an amino acid sequence being at least 80%, 85%, 90%, 95%, 96%, 97%, 98%, or 99% similar or identical (preferably identical) to SEQ ID NO: 50, and
   comprising or consisting of an amino acid sequence being at least 80%, 85%, 90%, 95%, 96%, 97%, 98%, or 99% similar or identical (preferably identical) to positions 44 to 49 of SEQ ID NO: 50, and
   comprising or consisting of an amino acid sequence being at least 80%, 85%, 90%, 95%, 96%, 97%, 98%, or 99% similar or identical (preferably identical) to positions 67 to 71 of SEQ ID NO: 50, and
   comprising or consisting of an amino acid sequence being at least 80%, 85%, 90%, 95%, 96%, 97%, 98%, or 99% similar or identical (preferably identical) to positions 108 to 122 of SEQ ID NO: 50.

In one embodiment of the present invention, the TCR as described and provided herein comprises
(A) at least one TCR alpha chain or subregion thereof according to the CDR3 alpha chain as described herein under (Aa), CDR½ alpha chain as described herein under (Aa1), the TCR variable alpha chain as described under (Aa2) or the TCR alpha chain as described under (Aa3), and at least one TCR beta chain or subregion thereof according to the CDR3 beta chain as described herein under (Ab), CDR½ beta chain as described herein under (Ab1), the TCR variable beta chain as described under (Ab2) or the TCR beta chain as described under (Ab3), covalently linked to each other to form a TCR heterodimer or multimer, or
(B) at least one TCR alpha chain or subregion thereof according to the CDR3 alpha chain as described herein under (Ba), CDR½ alpha chain as described herein under (Ba1), the TCR variable alpha chain as described under (Ba2) or the TCR alpha chain as described under (Ba3), and at least one TCR beta chain or subregion thereof according to the CDR3 beta chain as described herein under (Bb), CDR½ beta chain as described herein under (Bb1), the TCR variable beta chain as described under (Bb2) or the TCR beta chain as described under (Bb3), covalently linked to each other to form a TCR heterodimer or multimer.

In accordance with the present invention, the TCR as described and provided herein may be any kind of TCR. In one embodiment of the present invention, the TCR may be selected from the group consisting of a native TCR, a TCR variant, a TCR fragment, and a TCR construct. In a preferred embodiment of the present invention, the TCR is water soluble.

In accordance with the present invention, all TCR variants are preferably functional variants of the inventive TCR. The term “functional variant” as used herein refers to a TCR, polypeptide, or protein having substantial or significant sequence identity or similarity to a parent TCR, its variable regions or its antigen-binding regions and shares its biological activity, i.e. its ability to specifically bind to the antigenic target for which the parent TCR of the invention has antigenic specificity to a similar, the same or even a higher extent as the TCR disclosed herein and evaluated in the appended examples. Also encompassed by the present invention are TCR sequence variants.

The term “TCR variants” as used herein includes “sequence variants” of the TCR disclosed herein, i.e. variants substantially comprising the amino acid sequence of the inventive TCR as described above (also referred to as the “parent” TCR) but containing at least one amino acid modification (i.e. a substitution, deletion, or insertion) as compared to the “parent” TCR amino acid sequence, provided that the variant preferably retains the antigenic specificity of the inventive “parent” TCR. TCR sequence variants of the invention are typically prepared by introducing appropriate nucleotide changes into the nucleic acids encoding the “parent” TCR, or by peptide synthesis. Generally, the aforementioned amino acid modifications may be introduced into, or present in, the variable region or the constant region of the TCR and may serve to modulate properties like binding strength and specificity, post-translational processing (e.g. glycosylation), thermodynamic stability, solubility, surface expression or TCR assembly.

The term “TCR” as used herein further comprises TCR constructs. The term “construct” includes proteins or polypeptides comprising at least one antigen binding domain of the inventive TCR, but do not necessarily share the basic structure of a native TCR (i.e. variable domains incorporated into a TCR alpha chain and a TCR beta chain forming a heterodimer). TCR constructs and fragments are typically obtained by routine methods of genetic engineering and are often artificially constructed to comprise additional functional protein or polypeptide domains. In accordance with the foregoing, TCR constructs and fragments of the invention are envisaged to comprise at least one CDR3 alpha and/or at least one CDR3 beta as disclosed elsewhere herein. Further envisaged herein are constructs and fragments comprising at least one CDR1 alpha, CDR2 alpha, CDR1 beta, CDR2 beta, alpha chain variable region, beta chain variable region, alpha chain and/or beta chain, or combinations thereof, optionally in combination with further protein domains or moieties as exemplified herein. The TCR constructs and fragments provided herein are envisaged to be capable of specifically binding to the same antigenic target as the inventive TCR described above and evaluated in the appended Examples.

The TCR of the present invention encompasses heterodimers and multimers in which at least one TCR alpha chain variable region or TCR alpha chain and at least one TCR beta chain variable region are covalently linked to each other to form TCR heterodimers or multimers. A “multimer” as used in the present invention describes a molecule of diverse subunits or functional entities while a heterodimer comprises only two functional entities. In its simplest form a multivalent TCR construct according to the invention comprises a multimer of two or three or four or more TCRs associated (e.g. covalently or otherwise linked) with one another, preferably via a linker molecule. In this context “covalently linked” means a chemical bond between two molecules, sharing electron pairs describing a stable balance between atom bonds.

In accordance of the present invention, suitable linkers may have a spherical body, preferably a uniform bead, more preferably a polystyrene bead, most preferably a bio-compatible polystyrene bead. Such TCR constructs can also be comprised by an inventive TCR and a bead having a pre-defined fluorescence dye incorporated into the bead. Suitable linker molecules include, but are not limited to, multivalent attachment molecules such as avidin, streptavidin, neutravidin and extravidin, each of which has four binding sites for biotin. Thus, biotinylated TCRs can be formed into multimers having a plurality of TCR binding sites. The number of TCRs in the multimer will depend upon the quantity of TCR in relation to the quantity of linker molecule used to make the multimers, and also on the presence or absence of any other biotinylated molecules. Exemplary multimers are dimeric, trimeric, tetrameric or pentameric or higher-order multimer TCR constructs. Multimers of the invention may also comprise further functional entities such as labels or drugs or (solid) carriers.

In accordance of the present invention, a TCR heterodimer or multimer also relates to fusion proteins or polypeptides comprising at least one TCR alpha chain, TCR alpha chain variable region or CDR3 alpha and/or at least one TCR beta chain, TCR beta chain variable region or CDR3 beta; and further one or more fusion component(s). It may be at least one TCR alpha chain as defined herein and/ or at least one TCR beta chain as defined herein and/or an antibody or a single chain antibody fragment (scFv) which is directed against an antigen or epitope on the surface of lymphocytes, and also the TCR alpha chain(s) and TCR beta chain(s) are linked to each other and fused, optionally via a linker, to said antibody or scFv. Useful components include Fc receptors; Fc domains (derived from IgA, IgD, IgG, IgE, and IgM); cytokines (such as IL-2 or IL-15); toxins; antibodies or antigen-binding fragments thereof (such as anti-CD3, anti-CD28, anti-CD5, anti-CD16 or anti- CD56 antibodies or antigen-binding fragments thereof); CD247 (CD3-zeta), CD28, CD137, CD134 domains; or any combinations thereof.

Exemplary antibody fragments that can be used as fusion components in accordance with the present invention include fragments of full-length antibodies, such as (s)dAb, Fv, Fd, Fab, Fab′, F(ab′)2 or “r IgG” (“half antibody”); modified antibody fragments such as scFv, di-scFv or bi(s)-scFv, scFv-Fc, scFv-zipper, scFab, Fab2, Fab3, diabodies, single chain diabodies, tandem diabodies (Tandab’s), tandem di-scFv, tandem tri-scFv, minibodies, multibodies such as triabodies or tetrabodies, and single domain antibodies such as nanobodies or single variable domain antibodies comprising only one variable domain, which might be VHH, VH or VL.

TCR constructs of the invention may be fused to one or more antibody or antibody fragments, yielding monovalent, bivalent and polyvalent/multivalent constructs and thus monospecific constructs, specifically binding to only one target antigen as well as bispecific and polyspecific/multispecific constructs, which specifically bind more than one target antigens, e.g. two, three or more, through distinct antigen binding sites.

Optionally, a linker may be introduced between the one or more of the domains or regions of the TCR construct of the invention, i.e. between the TCR alpha chain CDR3, TCR alpha chain variable region, and/or a TCR alpha chain, the TCR beta chain CDR3, TCR beta chain variable region, and/or a TCR beta chain, and/or the one or more fusion component(s) described herein. Linkers are known in the art and have been reviewed, inter alia, by Chen et al., Adv Drug Deliv Rev. 2013 Oct. 15; 65(10): 1357-1369. In general, linkers include flexible, cleavable and rigid linkers and will be selected depending on the type of construct and intended use/application. For example, for therapeutic application, non-immunogenic, flexible linkers are often preferred in order to ensure a certain degree of flexibility or interaction between the domains while reducing the risk of adverse immunogenic reactions. Such linkers are generally composed of small, non-polar (e.g. Gly) or polar (e.g. Ser or Thr) amino acids and include “GS” linkers consisting of stretches of Gly and Ser residues.

Particularly useful TCR constructs envisaged in accordance with the invention are those comprising at least one TCR alpha chain, TCR alpha chain variable region or CDR3 alpha as defined herein, at least one TCR beta chain, TCR beta chain variable region or CDR3 beta as defined herein, optionally linked to each other and fused, optionally via a liker, to at least one antibody or an antibody fragment (such as a single chain antibody fragment (scFv)) directed against an antigen or epitope on the surface of lymphocytes. Useful antigenic targets recognized by the antibody or antibody fragment (e.g. scFv) include CD3, CD28, CD5, CD16 and CD56. Said construct can in general have any structure as long the “TCR portion” (i.e. TCR alpha and beta chain or variable regions or CDR3s thereof) retains its ability to recognize the antigenic target defined herein, and the “antibody portion” binds to the desired surface antigen or epitope, thereby recruiting and targeting the respective lymphocyte to the target cell. Such constructs may advantageously serve as “adapters” joining an antigen presenting cell displaying the antigenic target (such as a tumor cell) and a lymphocyte (such as a cytotoxic T cell or NK cell) together. An example of such a fusion protein is a construct engineered according to the principle of a bi-specific T cell engager (BiTE®) consisting of two single-chain variable fragments (scFvs) of different antibodies, on a single peptide chain of about 55 kilodaltons (kD). Accordingly, a TCR construct of the invention may comprise at least one TCR antigen binding domain as described herein (for instance a TCR variable alpha and variable beta chain fused to each other) linked to a scFv (or other binding domain) of the desired binding specificity, e.g. CD3 or CD56. The scFv (or other binding domain) binds to T cells such as via the CD3 receptor or to CD56 for NK cell activation, and the other to a tumor cell via an antigenic target specifically expressed on the tumor cell. Also envisaged herein are tribodies comprising at least one TCR antigen binding domain as described herein, an scFv (or other binding domain) and a further domain e.g. for targeting the construct to a site of action within the body (e.g. an Fc domain).

The TCR of the invention may be provided in “isolated” or “substantially pure” form. “Isolated” or “substantially pure” when used herein means that the TCR has been identified separated and/or recovered from a component of its production environment, such that the “isolated” TCR is free or substantially free of other contaminant components from its production environment that might interfere with its therapeutic or diagnostic use. Contaminant components may include enzymes, hormones, and other proteinaceous or non-proteinaceous solutes. “Isolated” TCRs will thus be prepared by a method for obtaining a TCR through incubating a host cell under conditions causing expression of said TCR, and purifying said TCR thus containing at least one purification step removing or substantially removing these contaminant components. The aforementioned definition is equally applicable to “isolated” polynucleotides/nucleic acids, mutatis mutandis.

The TCR of the present invention can be provided in soluble form. Soluble TCRs are useful as diagnostic tools, and carriers or “adapters” that specifically target therapeutic agents or effector cells to, for instance, a cancer cell expressing the antigenic target recognized by the soluble TCR. Soluble TCRs (sTCRs) will typically be fragments or constructs comprising TCR alpha and/or beta chains, or variable regions or CDRs thereof and optionally stabilized via disulfide bonds or covalently linked via a suitable linker molecule, e.g. as described above in the context of TCR constructs of the invention. They will typically not comprise e.g. a transmembrane region. In some circumstances, amino acid modifications in the polypeptide sequence may be introduced in order to enhance solubility of the molecules, and/or correct folding and pairing of the alpha and beta chains (if desired), in particular when produced in a recombinant host that does not provide for the aforementioned features. When using E. coli as production host cells for instance folding and pairing of the TCR alpha and beta chains is typically accomplished in vitro. A TCR according to the invention may therefore for instance comprise additional cysteine residues, as described elsewhere herein. In a preferred embodiment of the present invention, the TCR is water soluble.

Besides additional cysteine bridges, other useful modifications include, for instance, the addition of leucine zippers and/or ribosomal skipping sequences, e.g. sequence 2A from picorna virus as described in Walseng et al., (2015), PLoS ONE 10(4): e0119559 to increase folding, expression and/or pairing of the TCR alpha and/or beta chains.

The TCR of the invention may further comprise one or more modifications as described in the following. The modifications described below will typically be covalent modifications and can be accomplished using standard techniques known in the art. In some circumstances, amino acid modifications in the TCRs may be required in order to facilitate the introduction of said modifications.

In accordance with the present invention, the TCR as described and provided herein may further comprise one or more fusion component(s), e.g. those selected from Fc receptors; Fc domains, including IgA, IgD, IgG, IgE, and IgM; cytokines, including IL-2 or IL-15; toxins; antibodies or antigen-binding fragments thereof, including anti-CD3, anti-CD28, anti-CDS, anti-CD16 or anti-CD56 antibodies or antigen-binding fragments thereof; and CD247 (CD3-zeta), CD28, CD137, CD134 domain, or combinations thereof; optionally further comprising at least one linker.

In one embodiment of the present invention, the TCR as described and provided herein comprises
(A) at least one TCR alpha chain or subregion thereof according to the CDR3 alpha chain as described herein under (Aa), CDR½ alpha chain as described herein under (Aa1), the TCR variable alpha chain as described under (Aa2) or the TCR alpha chain as described under (Aa3), and at least one TCR beta chain or subregion thereof according to the CDR3 beta chain as described herein under (Ab), CDR½ beta chain as described herein under (Ab1), the TCR variable beta chain as described under (Ab2) or the TCR beta chain as described under (Ab3), optionally covalently linked to each other to form a TCR heterodimer or multimer, or
(B) at least one TCR alpha chain or subregion thereof according to the CDR3 alpha chain as described herein under (Ba), CDR½ alpha chain as described herein under (Ba1), the TCR variable alpha chain as described under (Ba2) or the TCR alpha chain as described under (Ba3), and at least one TCR beta chain or subregion thereof according to the CDR3 beta chain as described herein under (Bb), CDR½ beta chain as described herein under (Bb1), the TCR variable beta chain as described under (Bb2) or the TCR beta chain as described under (Bb3), optionally covalently linked to each other to form a TCR heterodimer or multimer,
   wherein the TCR further comprises an antibody or a single chain antibody fragment (scFv) which is directed against an antigen (e.g., CD3, CD28, CD5, CD16, or CD56) or epitope on the surface of lymphocytes,
   wherein the TCR alpha chain(s) or subregion thereof and TCR beta chain(s) or subregion thereof are linked to each other and fused, optionally via a linker, to said antibody or scFv.

The term “epitope” as used herein refers to a site on an antigen to which a recognition molecule (e.g., the TCR as described and provided herein) binds. Preferably, an epitope is a site on a molecule against which a recognition molecule, preferably a TCR or an antibody will be produced and/or to which a TCR or an antibody will bind. For example, an epitope can be recognized by a recognition molecule, particularly preferably by a TCR or an antibody defining the epitope. A “linear epitope” is an epitope where an amino acid primary sequence comprises the epitope recognized. A linear epitope typically includes at least 3, and more usually, at least 5, for example, about 8 to about 10 amino acids in a unique sequence.

In one embodiment of the present invention, the TCR as described and provided herein may further comprise at least one molecular marker.

The TCR, in particular (soluble) TCR, of the invention can be labelled with at least one molecular marker. Useful molecular markers are known in the art and can be coupled to the TCR or TCR variant using routine methods, optionally via linkers of various lengths.

In general, different marker fall into a variety of classes, depending on the assay in which they are to be detected - the following examples include, but are not limited to: isotopic marker, which may be radioactive or heavy isotopes, such as radioisotopes or radionuclides (e.g. <3>H, <14>, <15>N, <35>S, <89>Zr, <90>Y, <99>Tc, <111>In, <125>I, <131>I); magnetic marker (e.g. magnetic particles); redox active moieties; optical dyes (including, but not limited to, chromophores, phosphors and fluorophores) such as fluorescent groups (e.g. FITC, rhodamine, lanthanide phosphors), chemiluminescent groups, and fluorophores which can be either “small molecule” fluorophores or proteinaceous fluorophores; enzymatic groups (e.g. horseradish peroxidase, β-galactosidase, luciferase, alkaline phosphatase; biotinylated groups; or predetermined polypeptide epitopes recognized by a secondary reporter (e.g. leucine zipper pair sequences, binding sites for secondary antibodies, metal binding domains, epitope tags, etc.). Labelling with molecular markers is particularly envisaged when the TCR, TCR variants or especially soluble TCR constructs (such as those comprising at least one TCR alpha and/or TCR beta chain as described herein) are intended for diagnostic use.

The TCR, in particular soluble TCR, of the invention can be modified by attaching further functional moieties, e.g. for reducing immunogenicity, increasing hydrodynamic size (size in solution) solubility and/or stability (e.g. by enhanced protection to proteolytic degradation) and/or extending serum half-life.

Exemplary functional moieties for use in accordance with the invention include peptides or protein domains binding to other proteins in the human body (such as serum albumin, the immunoglobulin Fc region or the neonatal Fc receptor (FcRn), polypeptide chains of varying length (e.g. XTEN technology or PASylation®), non-proteinaceous polymers, including, but not limited to, various polyols such as polyethylene glycol (PEGylation), polypropylene glycol, polyoxyalkylenes, or copolymers of polyethylene glycol and polypropylene glycol, or of carbohydrates, such as hydroxyethyl starch (e.g. HESylation®) or polysialic acid (e.g. PolyXen® technology).

Other useful functional moieties include “suicide” or “safety switches” that can be used to shut off effector host cells carrying an inventive TCR in a patient’s body. An example is the inducible Caspase 9 (iCasp9) “safety switch” described by Gargett and Brown Front Pharmacol. 2014; 5: 235. Briefly, effector host cells are modified by well-known methods to express a Caspase 9 domain whose dimerization depends on a small molecule dimerizer drug such as AP1903/CIP, and results in rapid induction of apoptosis in the modified effector cells. The system is for instance described in EP2173869 (A2). Examples for other “suicide” “safety switches” are known in the art, e.g. Herpes Simplex Virus thymidine kinase (HSV-TK), expression of CD20 and subsequent depletion using anti-CD20 antibody or myc tags (Kieback et al., Proc Natl Acad Sci USA. 2008 Jan. 15;105(2):623-8). The inventive TCR can also be modified by introducing an inducible so called “on-switch” (as for example described in WO2019175209A1), wherein the modified alpha and beta chains of the inventive TCR only dimerize upon interaction with a small dimerizer drug subsequently resulting in a functional TCR which is only expressed on the cell surface in the presence of the dimerizer drug.

TCRs with an altered glycosylation pattern are also envisaged herein. As known in the art, glycosylation patterns can depend on the amino acid sequence (e.g. the presence or absence of particular glycosylation amino acid residues, discussed below) and/or the host cell or organism in which the protein is produced. Glycosylation of polypeptides is typically either N-linked or O-linked. N-linked refers to the attachment of the carbohydrate moiety to the side chain of an asparagine residue. Addition of N-linked glycosylation sites to the binding molecule is conveniently accomplished by altering the amino acid sequence such that it contains one or more tri-peptide sequences selected from asparagine-X-serine and asparagine-X-threonine (where X is any amino acid except proline). O-linked glycosylation sites may be introduced by the addition of or substitution by, one or more serine or threonine residues to the starting sequence.

Another means of glycosylation of TCRs is by chemical or enzymatic coupling of glycosides to the protein. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine.

Similarly, deglycosylation (i.e. removal of carbohydrate moieties present on the binding molecule) may be accomplished chemically, e.g. by exposing the TCRs to trifluoromethanesulfonic acid, or enzymatically by employing endo- and exo-glycosidases.

It is also conceivable to add a drug such as a small molecule compound to the TCR, in particular to the soluble TCR of the present invention. Linkage can be achieved via covalent bonds, or non-covalent interactions such as through electrostatic forces. Various linkers, known in the art, can be employed in order to form the drug conjugates.

The TCR, in particular soluble TCR, of the disclosure can be modified to introduce additional domains which aid in identification, tracking, purification and/or isolation of the respective molecule (tags). Non-limiting examples of such tags comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. Strep II-tag), His-tag, CD20, Her2/neu tags, myc-tag, FLAG-tag, T7-tag, HA(hemagglutinin)-tag, or GFP-tags.

Epitope tags are useful examples of tags that can be incorporated into the TCR of the disclosure. Epitope tags are short stretches of amino acids that allow for binding of a specific antibody and therefore enable identification and tracking of the binding and movement of soluble TCRs or host cells within the patient’s body or cultivated (host) cells. Detection of the epitope tag, and hence, the tagged TCR, can be achieved using a number of different techniques. Examples of such techniques include: immunohistochemistry, immunoprecipitation, flow cytometry, immunofluorescence microscopy, ELISA, immunoblotting (“Western”), and affinity chromatography. The epitope tags can for instance have a length of 6 to 15 amino acids, in particular 9 to 11 amino acids. It is also possible to include more than one epitope tag in the TCR of the invention.

Tags can further be employed for stimulation and expansion of host cells carrying an inventive TCR by cultivating the cells in the presence of binding molecules (antibodies) specific for said tag.

The present invention further relates to a nucleic acid encoding the TCR as described and provided herein. In specific embodiments of the present invention, such nucleic acid molecules may comprise a nucleic acid sequence being at least 80%, 85%, 90%, 95%, 96%, 97%, 98%, or 99% identical to the nucleic acid sequence of any one of SEQ ID NOs: 3, 5, 7, 9, 11, 13, 15, 17, 19, or 21; or being at least 80%, 85%, 90%, 95%, 96%, 97%, 98%, or 99% identical to the nucleic acid sequence of any one of SEQ ID NOs: 31, 33, 35, 37, 39, 41, 43, 45, 47, or 49.

As used herein, unless specifically defined otherwise, the term “nucleic acid” or “nucleic acid molecule” is used synonymously with “oligonucleotide”, “nucleic acid strand”, or the like, and means a polymer comprising one, two, or more nucleotides, e.g., single- or double stranded.

Generally, as used herein, the terms “polynucleotide”, “nucleic acid″ and “nucleic acid molecule” are to be construed synonymously. Generally, nucleic acid molecules may comprise inter alia DNA molecules (such as dsDNA, ssDNA, cDNA), RNA molecules (such as dsRNA, ssRNA, mRNA ivtRNA), oligonucleotide thiophosphates, substituted ribo-oligonucleotides or PNA molecules. Furthermore, the term “nucleic acid molecule” may refer to DNA or RNA or hybrids thereof or any modification thereof that is known in the art (see, e.g., US 5525711, US 471 1955, US 5792608 or EP 302175 for examples of modifications). The polynucleotide sequence may be single- or double- stranded, linear or circular, natural or synthetic, and without any size limitation. For instance, the polynucleotide sequence may be genomic DNA, cDNA, mitochondrial DNA, mRNA, antisense RNA, ribozymal RNA or a DNA encoding such RNAs or chimeroplasts (Gamper, Nucleic Acids Research, 2000, 28, 4332 -4339). Said polynucleotide sequence may be in the form of a vector, plasmid or of viral DNA or RNA. Also described herein are nucleic acid molecules which are complementary to the nucleic acid molecules described above and nucleic acid molecules which are able to hybridize to nucleic acid molecules described herein. A nucleic acid molecule described herein may also be a fragment of the nucleic acid molecules in context of the present invention. Particularly, such a fragment is a functional fragment. Examples for such functional fragments are nucleic acid molecules which can serve as primers.

The present invention further relates to a vector comprising a nucleic acid molecule as described and provided herein.

The term “vector” as used herein particularly refers to plasmids, cosmids, viruses, bacteriophages and other vectors commonly used in genetic engineering. In one embodiment of the present invention, the vectors are suitable for the transformation, transduction and/or transfection of host cells as described herein, e.g., prokaryotic cells (e.g., (eu)bacteria, archaea), eukaryotic cells (e.g., mammalian cells, insect cells) fungal cells, yeast, and the like. Examples of bacterial host cells in context with the present invention comprise Gram negative and Gram positive cells. Preferably, the host cells are eukaryotic cells, e.g., human cells. Specific examples for suitable host cells may comprise inter alia lymphoblastoid cell lines, cytotoxic T lymphocytes (CTLs), CD8+ T cells (preferably autologous CD8+ cells), CD4+ T cells (preferably autologous CD4+ cells), T memory stem cells (T_{SCM}), natural killer (NK) cells (e.g., modified to recombinantly express CD3 (including CD3 gamma, CD3 delta, CD3 epsilon), as also described and provided in WO2016/116601)), natural killer T (NKT) cells, and gamma/ delta-T cells. In one embodiment of the present invention, said vectors are suitable for stable transformation of the host cells.

Accordingly, in one aspect of the invention, the vector as provided is an expression vector. Generally, expression vectors have been widely described in the literature. As a rule, they may not only contain a selection marker gene and a replication-origin ensuring replication in the host selected, but also a promoter, and in most cases a termination signal for transcription. Between the promoter and the termination signal there is preferably at least one restriction site or a polylinker which enables the insertion of a nucleic acid sequence/molecule desired to be expressed. It is to be understood that when the vector provided herein is generated by taking advantage of an expression vector known in the prior art that already comprises a promoter suitable to be employed in context of this invention. The nucleic acid construct is preferably inserted into that vector in a manner the resulting vector comprises only one promoter suitable to be employed in context of this invention. The skilled person knows how such insertion can be put into practice. For example, the promoter can be excised either from the nucleic acid construct or from the expression vector prior to ligation. In one embodiment of the present invention, the vector is able to integrate into the host cell genome. The vector may be any vector suitable for the respective host cell, preferably an expression vector. In context with the present invention, preferred vectors include lentiviral and retroviral vectors as known in the art.

Besides an origin of replication, selection markers, and restriction enzyme cleavage sites, expression vectors typically include one or more regulatory sequences operably linked to the heterologous polynucleotide to be expressed.

The term “regulatory sequence” refers to a nucleic acid sequence necessary for the expression of an operably linked coding sequence of a (heterologous) polynucleotide in a particular host organism or host cell and thus include transcriptional and translational regulatory sequences. Typically, regulatory sequences required for expression of heterologous polynucleotide sequences in prokaryotes include a promoter(s), optionally operator sequence(s), and ribosome binding site(s). In eukaryotes, promoters, polyadenylation signals, enhancers and optionally splice signals are typically required. Moreover, specific initiation and secretory signals also may be introduced into the vector in order to allow for secretion of the polypeptide of interest into the culture medium.

A nucleic acid is “operably linked” when it is placed into a functional relationship with another nucleic acid sequence, in particular on the same polynucleotide molecule. For example, a promoter is operably linked with a coding sequence of a heterologous gene when it is capable of effecting the expression of that coding sequence. The promoter is typically placed upstream of the gene encoding the polypeptide of interest and regulates the expression of said gene.

Exemplary regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. As set out before, the expression vectors may also include origins of replication and selectable markers.

In accordance with the present invention, particularly retroviral and lentiviral vectors are useful. Examples for suitable expression vectors include viral vectors, such as lentiviral or retroviral vectors e.g. MP71 vectors or retroviral SIN vectors; and lentiviral vectors or lentiviral SIN vectors. Viral vectors comprising polynucleotides encoding the TCR of the invention are for instance capable of infecting lymphocytes, which are envisaged to subsequently express the heterologous TCR. Another example for a suitable expression vector is the Sleeping Beauty (SB) transposon transposase DNA plasmid system, SB DNA plasmid. The nucleic adds and/or in particular expression constructs of the invention can also be transferred into cells by transient RNA transfection.

Currently used viral vectors for native TCR expression typically link the TCR-alpha and TCR-beta chain genes in one vector with either an internal ribosomal entry site (IRES) sequence or the 2A peptide sequence derived from a porcine tsechovirus, resulting in the expression a single messenger RNA (mRNA) molecule under the control of the viral promoter within the transduced cell.

The present invention further relates to a host cell comprising the TCR as described and provided herein, a nucleic acid molecule as described and provided herein, or a vector as described and provided herein. A variety of host cells can be used in accordance with the invention. As used herein, the term “host cell” encompasses cells which can be or has/have been recipients of polynucleotides or vectors described herein and/or express (and optionally secreting) the TCR of the present invention. The terms “cell” and “cell culture” are used interchangeably to denote the source of a TCR unless it is clearly specified otherwise. The term “host cell” also includes host cell lines. In general, the term “host cell” includes prokaryotic or eukaryotic cells, and also includes without limitation bacteria, yeast cells, fungi cells, plant cells, and animal cells such as insect cells and mammalian cells, e.g. murine, rat, macaque or human cells. The invention thus provides, inter alia, host cells comprising a polynucleotide or a vector, e.g. an expression vector comprising a nucleotide sequence encoding a TCR or TCR construct as described herein. Polynucleotides and/or vectors of the invention can be introduced into the host cells using routine methods known in the art, e.g. by transfection, transformation, or the like.

“Transfection” is the process of deliberately introducing nucleic acid molecules or polynucleotides (including vectors) into target cells. An example is RNA transfection, i.e. the process of introducing RNA (such as in vitro transcribed RNA, ivtRNA) into a host cell. The term is mostly used for non-viral methods in eukaryotic cells. The term “transduction” is often used to describe virus-mediated transfer of nucleic acid molecules or polynucleotides. Transfection of animal cells typically involves opening transient pores or “holes” in the cell membrane, to allow the uptake of material. Transfection can be carried out using calcium phosphate, by electroporation, by cell squeezing or by mixing a cationic lipid with the material to produce liposomes, which fuse with the cell membrane and deposit their cargo inside. Exemplary techniques for transfecting eukaryotic host cells include lipid vesicle mediated uptake, heat shock mediated uptake, calcium phosphate mediated transfection (calcium phosphate/DNA co-precipitation), microinjection and electroporation.

“Transformation” is used to describe non-viral transfer of nucleic acid molecules or polynucleotides (including vectors) into bacteria, and also into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules). Transformation can be effected by artificial means. For transformation to happen, cells or bacteria must be in a state of competence, which might occur as a time-limited response to environmental conditions such as starvation and cell density. For prokaryotic transformation, techniques can include heat shock mediated uptake, bacterial protoplast fusion with intact cells, microinjection and electroporation. Techniques for plant transformation include Agrobacterium mediated transfer, such as by A. tumefaciens, rapidly propelled tungsten or gold microprojectiles, electroporation, microinjection and polyethylene glycol mediated uptake.

In accordance with the present invention, for expression of the TCR of the invention, a host cell may be chosen that modulates the expression of the inserted polynucleotide sequences, and/or modifies and processes the gene product (i.e. RNA and/or protein) as desired. Such modifications (e.g. glycosylation) and processing (e.g. cleavage) of gene products may be important for the function of the TCR. Different host cells have characteristic and specific mechanisms for the post-translational processing and modification of gene products. Appropriate cell lines or host systems can be chosen to ensure the correct modification and processing of the product. To this end, eukaryotic host cells that possess the cellular machinery for proper processing of the primary transcript, glycosylation, and phosphorylation of the gene product may be used

In accordance with the present invention, a host cell comprising the TCR as described and provided herein, a nucleic acid molecule as described and provided herein, or a vector as described and provided herein may be any cell which is suitable to stably express a TCR as described and provided herein. Preferably, such host cell is able to present such TCR on its surface, allowing (specific) binding of said TCR to a PRAME_{L-L}-peptide as described and specified herein (or a portion thereof, or in its HLA-A bound form as described and specified herein).

Host cells in accordance with the present invention may be “production host cells” used for the expression of a soluble TCR of the invention and are preferably capable of expressing high amounts of recombinant protein. In accordance with the foregoing, conceivable expressions systems (i.e. host cells comprising an expression vector as described above) include microorganisms such as bacteria (e.g. E. coli, B. subtilis) transformed with recombinant bacteriophage DNA, plasmid DNA or cosmid DNA expression vectors; yeast (e.g. Saccharomyces, Pichia) transformed with recombinant yeast expression vectors; insect cell systems infected with recombinant virus expression vectors (e.g. baculovirus); plant cell systems infected with recombinant virus expression vectors (e.g. cauliflower mosaic virus, CaMV; tobacco mosaic virus, TMV) or transformed with recombinant plasmid expression vectors (e.g., Ti plasmid). Mammalian expression systems harboring recombinant expression constructs containing promoters derived from the genome of mammalian cells (e.g. metallothionein promoter) or from mammalian viruses (e.g. the adenovirus late promoter; the vaccinia virus 7.5 K promoter, the cytomegalovirus (CMV) major immediate-early promoter (MIEP) promoter) are often preferred. Suitable mammalian host cells can be selected from known cell lines (e.g. COS, CHO, BLK, 293, 3T3 cells), however it is also conceivable to use lymphocytes such as cytotoxic T lymphocytes (CTLs), CD8+ T cells, CD4+ T cells, natural killer (NK) cells, natural killer T (NKT) cells, gamma/ delta-T-cells.

Exemplary mammalian host cells that can be used for as “production host cells” include Chinese Hamster Ovary (CHO cells) including DHFR minus CHO cells such as DG44 and DUXBI 1, NSO, COS (a derivative of CVI with SV40 T antigen), HEK293 (human kidney), and SP2 (mouse myeloma) cells. Other exemplary host cell lines include, but are not limited to, HELA (human cervical carcinoma), CVI (monkey kidney line), VERY, BHK (baby hamster kidney), MDCK, 293, W138, R1610 (Chinese hamster fibroblast) BALBC/3T3 (mouse fibroblast), HAK (hamster kidney line), P3×63-Ag3.653 (mouse myeloma), BFA-IcIBPT (bovine endothelial cells), and RAJI (human lymphocyte). Host cell lines are typically available from commercial services, the American Tissue Culture Collection (ATCC) or from published literature. Non-mammalian cells such as bacterial, yeast, insect or plant cells are also readily available and can also be used as “production host cells” as described above. Exemplary bacterial host cells include Enterobacteriaceae, such Escherichia coli, Salmonella; Bacillaceae, such as Bacillus subtilis; Pneumococcus; Streptococcus, and Haemophilus influenza. Other host cells include yeast cells, such as Saccharomyces cerevisiae, and Pichia pastoris. Insect cells include, without limitation, Spodoptera frugiperda cells.

In accordance with the foregoing, the present invention also provides a method for producing and obtaining a TCR as described herein comprising the steps of (a) incubating a host cell (i.e. a production host cell) under conditions causing expression of said TCR and (b) purifying said TCR.

The host cells harboring the expression vector are grown under conditions appropriate for the production of the TCR provided herein, in particular alpha chains and/or beta chains as described elsewhere herein, and assayed for alpha and/or beta chain protein synthesis. For the expression of double-chained TCRs, vectors encoding both the alpha and beta chains may be co-expressed in the host cell for expression of the entire molecule. Once a TCR of the invention has been expressed, it may be purified by any purification method known in the art, for example, by chromatography (e.g. ion exchange chromatography (e.g. hydroxylapatite chromatography), affinity chromatography, particularly Protein A, Protein G or lectin affinity chromatography, sizing column chromatography), centrifugation, differential solubility, hydrophobic interaction chromatography, or by any other standard technique for the purification of proteins. The skilled person will readily be able to select a suitable purification method based on the individual characteristics of the TCR to be recovered.

The host cell described and provided in context with the present invention may also be an “effector host cells” comprising a nucleotide sequence, vector or TCR of the invention. Said effector host cells are modified using routine methods to comprise a nucleic acid sequence encoding the TCR of the invention, and are envisaged to express the TCR described herein, in particular on the cell surface. For the purposes of the present invention, “modified host cells expressing a TCR of the invention” generally refers to (effector or production) host cells treated or altered to express a TCR according to the present invention, for instance by RNA transfection as described in the appended Examples. Other methods of modification or transfection or transduction, such as those described elsewhere herein, are also envisaged. The term “modified host cell” thus includes “transfected”, “transduced” and “genetically engineered” host cells preferably expressing the TCR of the present invention. Preferably, such “(modified) effector host cells” (in particular “(modified) effector lymphocytes”) are capable of mediating effector functions through intracellular signal transduction upon binding of the TCR to its specific antigenic target. Such effector functions include for instance the release of perforin (which creates holes in the target cell membrane), granzymes (which are proteases that act intracellularly to trigger apoptosis), the expression of Fas ligand (which activates apoptosis in a FAS-bearing target cell) and the release of cytokines, preferably Th1/Tc1 cytokines such as IFN-gamma, IL-2 and TNF-α. Thus, an effector host cell engineered to express the TCR of the invention that is capable recognizing and binding to its antigenic target in the subject to be treated is envisaged to carry out the above-mentioned effector functions, thereby killing the target (e.g. cancer) cells. Cytolysis of target cells can be assessed e.g. with the CTL fluorescent killing assay (CTL, USA) detecting the disappearance of fluorescently labeled target cells during co-culture with TCR-transfected recipient T cells.

In view of the above, effector host cells preferably express a functional TCR, i.e. that typically comprises a TCR alpha and beta chain described herein; and also the signal transducing subunits CD3 gamma, delta, epsilon and zeta (CD3 complex). Moreover, expression of co-receptors CD4 or CD8 may also be desired. Generally, lymphocytes harboring the required genes involved in antigen binding, receptor activation and downstream signaling (e.g. Lck, FYN, CD45, and/or Zap70), T cells are particularly suitable as effector host cells. However, effector host cells expressing the TCR of the invention as a “binding domain” without the CD3 signal transducing subunit and/or aforementioned downstream signaling molecules (i.e. being capable of recognizing the antigenic target described herein, but without effecting functions mediated by CD3 and/or the aforementioned downstream signaling molecules) are also envisaged herein. Such effector cells are envisaged to be capable of recognizing the antigenic target described herein, and optionally of effecting other functions not associated with CD3 signaling and/or signaling of the aforementioned downstream signaling molecules. Examples include NK or NKT cells expressing the inventive TCR and being capable of e.g. releasing cytotoxic granules upon recognition of their antigenic target.

Thus, cytotoxic T lymphocytes (CTLs), CD8+ T cells, CD4+ T cells, natural killer (NK) cells, natural killer T (NKT) cells, gamma/delta-T cells are considered useful lymphocyte effector host cells. Such lymphocytes expressing the recombinant TCR of the invention are also referred to as “modified effector lymphocytes” herein. The skilled person will however readily acknowledge that in general any component of the TCR signaling pathway leading to the desired effector function can be introduced into a suitable host cell by recombinant genetic engineering methods known in the art. Effector host cells in particular lymphocytes such as T cells can be autologous host cells that are obtained from the subject to be treated and transformed or transduced to express the TCR of the invention. Typically, recombinant expression of the TCR will be accomplished by using a viral vector as described in the appended Examples. Techniques for obtaining and isolating the cells from the patient are known in the art.

As mentioned earlier, the effector host cells provided herein are particularly envisaged for therapeutic applications. Further genetic modifications of the host cells may be desirable in order to increase therapeutic efficacy. E.g. when using autologous CD8+ T cells as “effector host cells” suitable additional modifications include downregulation of the endogenous TCR, CTLA-4 and/or PD-1 expression; and/or amplification of co-stimulatory molecules such as CD28, CD134, CD137. Means and methods for achieving the aforementioned genetic modifications have been described in the art.

Methods for targeted genome engineering of host cells are known in the art and include, besides gene knockdown with siRNA, the use of so-called “programmable nucleases” such as zinc-finger nucleases (ZFNs), transcription activator-like effector nucleases (TALENs) and RNA-guided engineered nucleases (RGENs) derived from the bacterial clustered regularly interspaced short palindromic repeat (CRISPR)-Cas (CRISPR-associated) system, as inter alia reviewed in Kim & Kim Nature Reviews Genetics 15, 321-334 (2014). For instance, programmable nucleases such as TALENs can be employed to cut the DNA regions that code for “unwanted” proteins, such as PD-1, CTLA-4 or an endogenous TCR, and thereby reducing their expression. When T cells are used as (effector) host cells, downregulation of the endogenous TCR has the benefit of reducing unwanted “mispairing” of endogenous and exogenous TCR alpha/beta chains.

In particular embodiments of the present invention, such host cells may be, e.g., selected from lymphocytes including but not limited to lymphoblastoid cell lines, cytotoxic T lymphocytes (CTLs), CD8+ T cells (preferably autologous CD8+ cells), CD4+ T cells (preferably autologous CD4+ cells), T memory stem cells (T_{SCM}), natural killer (NK) cells (e.g., modified to recombinantly express CD3 (including CD3 gamma, CD3 delta, CD3 epsilon), as also described and provided in WO2016/116601)), natural killer T (NKT) cells, and gamma/ delta-T cells.

The present invention further relates to a method for obtaining a TCR as described and provided herein, comprising incubating a host cell as described and provided herein under conditions causing expression of said TCR, and purifying said TCR.

The present invention further relates to a pharmaceutical or diagnostic composition comprising one or more of:
(i) a TCR as described and provided herein;
(ii) a nucleic acid molecule as described and provided herein;
(iii) a vector as described and provided herein; and/or
(iv) a host cell as described and provided herein, and
optionally pharmaceutically excipient(s).

The term “pharmaceutical composition” particularly refers to a composition suitable for administering to a human. However, compositions suitable for administration to non-human animals are generally also encompassed by the term.

The pharmaceutical composition envisaged by the present invention may further comprise one or more checkpoint inhibitors, preferably selected from the group consisting of a CTLA-4 inhibitor, a PD-1 inhibitor and a PD-L1 inhibitor. All of the above-mentioned inhibitors are immune checkpoint inhibitors capable of immune response downregulation. The cytotoxic lymphocyte-associated protein 4 (CTLA-4) inhibitor is a constitutively expressed protein receptor in regulatory T cells, but only upregulated in conventional T cells after activation. PD-1 and PD-L1 inhibitors act to inhibit the association of the programmed death-ligand 1 (PD-L1) with its receptor, programmed cell death protein 1 (PD-1). The interaction of these cell surface proteins is involved in the suppression of the immune system and occurs following infection to limit the killing of bystander host cells and prevent autoimmune disease. It thus is preferred that said checkpoint inhibitors are combined to the pharmaceutical composition according to in the present invention.

In accordance with the present invention, the pharmaceutical composition as described and provided herein may further comprise a checkpoint inhibitor. In one embodiment of the present invention, said checkpoint inhibitor may be selected from the group consisting of a CTLA-4 inhibitor, a PD-1 inhibitor and a PD-L1 inhibitor.

Further checkpoint inhibitors encompassed by the present invention are LAG3, ICOS, TIM3, VISTA and CEACAM1. LAG3 is an Inhibitory receptor on antigen activated T-cells. The ICOS protein belongs to the CD28 and CTLA-4 cell-surface receptor family. It forms homodimers and plays an important role in cell-cell signalling, immune responses, and regulation of cell proliferation. TIM3 or Hepatitis A Virus Cellular Receptor encodes a protein belonging to the immunoglobulin superfamily, and TIM family of proteins. CD4-positive T helper lymphocytes can be divided into types 1 (Th1) and 2 (Th2) on the basis of their cytokine secretion patterns. VISTA or V-Set Immunoregulatory Receptor encodes an immunoregulatory receptor which inhibits T-cell response. The CEACAM1 gene encodes a member of the carcinoembryonic antigen (CEA) gene family, which belongs to the immunoglobulin superfamily. These checkpoint inhibitors may also be combined with the pharmaceutical composition.

The pharmaceutical composition and its components (i.e. active agents and optionally excipients) are preferably pharmaceutically acceptable, i.e. capable of eliciting the desired therapeutic effect without causing any undesirable local or systemic effects in the recipient. Pharmaceutically acceptable compositions of the invention may for instance be sterile. Specifically, the term “pharmaceutically acceptable” may mean approved by a regulatory agency or other generally recognized pharmacopoeia for use in animals, and more particularly in humans.

The active agent described in the foregoing (for instance the host cell or the TCR) is preferably present in the pharmaceutical composition in a therapeutically effective amount. By “therapeutically effective amount” is meant an amount of the active agent that elicits the desired therapeutic effect. Therapeutic efficacy and toxicity can be determined by standard procedures, e.g. in cell culture or in test animals, e.g. ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred.

The exact dosage of the TCR polynucleotide, vector or host cell will be ascertainable by one skilled in the art using known techniques. Suitable dosages provide sufficient amounts of the active agent of the invention and are preferably therapeutically effective, i.e. elicit the desired therapeutic effect.

As is known in the art, adjustments for purpose of the treatment (e.g. remission maintenance vs. acute flare of disease), route, time and frequency of administration, time and frequency of administration formulation, age, body weight, general health, sex, diet, severity of the disease state, drug combination(s), reaction sensitivities, and tolerance/response to therapy may be necessary. Suitable dosage ranges, for instance for a soluble TCR as described herein, can be determined using data obtained from cell culture assays and animal studies and may include the ED₅₀. Typically, dosage amounts may vary from 0.1 to 100000 micrograms, up to a total dose of about 2 g, depending upon the route of administration. Exemplary dosages of the active agent of the invention are in the range from about 0.01 mg/kg to about 10 mg/kg, from about 0.1 mg/kg to about 10 mg/kg, from about 1 mg/kg to about 10 mg/kg, from about 1 mg/kg to about 5 mg/kg, from about 0.01 mg/kg to about 1 mg/kg, or from about 0.1 mg/kg to about 1 mg/kg. Guidance as to particular dosages and methods of delivery is provided in the literature. It is recognized that treatment may require a single administration of a therapeutically effective dose, or multiple administrations of a therapeutically effective dose of the active agent of the invention. E.g., some pharmaceutical compositions might be administered every 3 to 4 days, every week, or once every two weeks, or once within a month depending on formulation, half-life and clearance rate of the particular composition. As set out previously, the pharmaceutical composition may optionally comprise one or more excipients and/or additional active agents.

The term “excipient” includes fillers, binders, disintegrants, coatings, sorbents, anti-adherents, glidants, preservatives, antioxidants, flavoring, coloring, sweeting agents, solvents, co-solvents, buffering agents, chelating agents, viscosity imparting agents, surface active agents, diluents, humectants, carriers, diluents, preservatives, emulsifiers, stabilizers and tonicity modifiers. It is within the knowledge of the skilled person to select suitable excipients for preparing the desired pharmaceutical composition of the invention. Exemplary carriers for use in the pharmaceutical composition of the invention include saline, buffered saline, dextrose, and water. Typically, choice of suitable excipients will inter alia depend on the active agent used, the disease to be treated, and the desired formulation of the pharmaceutical composition.

The present invention further provides pharmaceutical compositions comprising one or more of the inventive active agents specified above (for instance a host cell or a TCR construct), and one or more additional active agents that are suitable for treatment and/or prophylaxis of the disease to be treated. Preferred examples of active ingredients suitable for combinations include known anti-cancer drugs such as cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolmide, topotecan, trimetreate glucuronate, auristatin E vincristine and doxorubicin; and peptide cytotoxins such as ricin, diphtheria toxin, pseudomonas bacterial exotoxin A, DNAase and RNAase; radio-nuclides such as iodine 131, rhenium 186, indium 111, yttrium 90, bismuth 210 and 213, actinium 225 and astatine 213; prodrugs, such as antibody directed enzyme pro-drugs; immuno-stimulants, such as IL-2, chemokines such as IL-8, platelet factor 4, melanoma growth stimulatory protein, etc., antibodies or fragments thereof such as anti-CD3 antibodies or fragments thereof, complement activators, xenogeneic protein domains, allogeneic protein domains, viral/bacterial protein domains and viral/bacterial peptides.

A variety of routes are applicable for administration of the pharmaceutical composition according to the present invention. Typically, administration will be accomplished parentally. Methods of parenteral delivery include topical, intra-arterial, intramuscular, subcutaneous, intramedullary, intrathecal, intraventricular, intravenous, intraperitoneal, intrauterine, intravaginal, sublingual or intranasal administration.

The pharmaceutical compositions of the invention can be formulated in various forms, depending inter alia on the active agent used (e.g. soluble TCR), e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for the desired method of administration. Processes known per se for producing medicaments are indicated in 22nd edition of Remington’s Pharmaceutical Sciences (Ed. Maack Publishing Co, Easton, Pa., 2012) and may include, for instance conventional mixing, dissolving, granulating, dragee-making, levigating, emulsifying, encapsulating, entrapping or lyophilizing processes. Pharmaceutical compositions comprising, for instance, host cells or soluble TCR as described herein will typically be provided in a liquid form, and preferably comprise a pharmaceutically acceptable buffer.

After pharmaceutical compositions of the invention have been prepared, they can be placed in an appropriate container and labeled for treatment of an indicated condition. Such labeling would for instance include amount, frequency and method of administration. In view of the foregoing the present invention thus provides a TCR, nucleic acid, vector and/or host cell as described herein for use as a medicament in detection, diagnosis, prognosis, prevention and/or treatment of cancer.

The TCR, nucleic acid, vector and/or host cell can in general be employed for treatment detection, diagnosis, prognosis, prevention and/or treatment of diseases or disorders. The term “treatment” in all its grammatical forms includes therapeutic or prophylactic treatment of a subject in need thereof. A “therapeutic or prophylactic treatment” comprises prophylactic treatments aimed at the complete prevention of clinical and/or pathological manifestations or therapeutic treatment aimed at amelioration or remission of clinical and/or pathological manifestations. The term “treatment” thus also includes the amelioration or prevention of diseases.

Such diseases envisaged to be treated when using the pharmaceutical composition of the present invention are preferably cancer selected from the group consisting of melanoma, bladder carcinoma, colon carcinoma, and breast adenocarcinoma, sarcoma, prostate cancer, uterine cancer, uveal cancer, uveal melanoma, squamous head and neck cancer, synovial carcinoma, Ewing’s sarcoma, triple negative breast cancer, thyroid cancer, testicular cancer, renal cancer, pancreatic cancer, ovarian cancer, esophageal cancer, non-small-cell lung cancer, non-Hodgkin’s lymphoma, multiple myeloma, melanoma, hepatocellular carcinoma, head and neck cancer, gastric cancer, endometrial cancer, colorectal cancer, cholangiocarcinoma, breast cancer, bladder cancer, myeloid leukemia and acute lymphoblastic leukemia, preferably wherein the cancer is selected from the group consisting of NSCLC, SCLC, breast, ovarian or colorectal cancer, sarcoma or osteosarcoma.

The terms “subject” or “individual” or “animal” or “patient” are used interchangeably herein to refer to any subject, particularly a mammalian subject, for whom therapy is desired. Mammalian subjects generally include humans, non-human primates, dogs, cats, guinea pigs, rabbits, rats, mice, horses, cattle, cows, and the like. However, it will readily be understood that the TCR, nucleic acids, vectors, host cells and pharmaceutical compositions provided herein are especially envisaged for treatment of human subjects, in particular those that are HLA-A2-positive.

For therapy, a TCR - in particular a soluble TCR of the invention -, nucleic acids, vectors (such as viral vectors) or host cells of the invention can be administered directly to the subject in need thereof. Thus, the present invention provides a TCR, nucleic acid, vector or host cells for use in a method of detecting, diagnosing, prognosing, preventing and/or treating of cancer. Said method can comprise the steps of (a) providing one or more of (i) a TCR (ii), a nucleic acid, (iii) a vector, (iv) a host cell, and/or (v) a pharmaceutical composition of the present invention; and (b) administering one or more of (i)-(v) to the subject in need thereof. Optionally, the method can comprise a further step of cancer therapy, e.g. radiation, or administration of one or more anti-cancer agents.

Treatment according to the invention may also comprise the steps of (a) providing a sample of a subject, said sample comprising lymphocytes; (b) providing one or more of (i) the TCR, (ii) nucleic acid, (ii) vector (iv) host cell and/or (v) pharmaceutical composition of the invention (c) introducing of one or more of (i) to (v) of step (b) into the lymphocytes of step (a) and, thereby, obtaining modified lymphocytes, (d) administering the modified lymphocytes of step (c) to a subject or patient in need thereof. The lymphocytes provided in step (a) are particularly envisaged to be “effector host cells” as described in the foregoing and are advantageously selected from T cells, NK cells and/or NKT cells, especially CD8⁺ T cells; and can be obtained in a previous step from a sample - in particular a blood sample - of the subject by routine methods known in the art. It is however also conceivable to use other lymphocytes that are preferably capable of expressing the TCR of the present invention and exert the desired biological effector functions as described herein. Moreover, said lymphocytes will typically be selected for compatibility with the subject’s immune system, i.e. they will preferably not elicit an immunogenic response. For instance, it is conceivable to use a “Universal Recipient cells”, i.e. universally compatible lymphocytes exerting the desired biological effector functions that can be grown and expanded in vitro. Use of such cells will thus obviate the need for obtaining and providing the subject’s own lymphocytes in step (a). The ex vivo introduction of step (c) can be carried out by introducing a nucleic acid or vector described herein via electroporation into the lymphocytes, or by infecting the lymphocytes with a viral vector, such as a lentiviral or retroviral vector as described previously in the context of the effector host cell. Other conceivable methods include the use of by transfection reagents, such as liposomes, or transient RNA transfection. The transfer of antigen-specific TCR genes into (primary) T cells by e.g. (retro-)viral vectors or transient RNA transfection represents a promising tool for generating tumor-associated antigen-specific T cells that can subsequently be re-introduced into the donor, where they specifically target and destroy tumor cells expressing said antigen. In the present invention, said tumor-associated antigen is PRAME as defined herein, particularly in its HLA-A*24 or HLA-A*02:17 bound form.

Treatment according to the invention may also comprise the steps of (a) providing a sample of a subject, said sample comprising lymphocytes; while the treatment consists of (b) providing one or more of (i) the TCR; (ii) the nucleic acid;(iii) the vector; (iv) the host cell; and (v) the pharmaceutical composition; (c) introducing of one or more of (i) to (v) of step (b) into the lymphocytes of step and, thereby, obtaining modified lymphocytes, (d) administering the modified lymphocytes of step (c) to a subject or patient in need thereof.

In view of the above, a further aspect of the present invention is thus the use of a TCR, a nucleic acid sequence, a vector and/or a host cell as described elsewhere herein for generating modified lymphocytes. Means and methods for introducing, e.g. a nucleic acid and a vector into the lymphocytes have been described elsewhere herein.

The present invention also provides a diagnostic composition comprising, as one or more diagnostic agent(s), the TCR, nucleic acid, the vector and/or the host cell as described herein. Typically, said diagnostic agent will comprise means for detecting its binding to its antigenic target, for instance a label as described in the context of the TCR constructs of the invention. As regards the host cell, it is for instance conceivable to use modified host cells comprising a dye or a contrast agent that is released (instead of cytotoxic granules) upon antigen recognition.

The present invention further relates to the TCR as described and provided herein, the nucleic acid molecule as described and provided herein, the vector as described and provided herein and/or the host cell as described and provided herein for use as a medicament.

The present invention further relates to the TCR as described and provided herein, the nucleic acid molecule as described and provided herein, the vector as described and provided herein and/or the host cell as described and provided herein for use in detection, diagnosis, prognosis, prevention and/or treatment of cancer. In context with the present invention, in specific embodiments, the cancer may be selected from the group consisting of melanoma, bladder carcinoma, colon carcinoma, breast adenocarcinoma, sarcoma, prostate cancer, uterine cancer, uveal cancer, uveal melanoma, squamous head and neck cancer, synovial carcinoma, Ewing’s sarcoma, triple negative breast cancer, thyroid cancer, testicular cancer, renal cancer, pancreatic cancer, ovarian cancer, esophageal cancer, non-small-cell lung cancer (NSCLC), small-cell lung cancer (SCLC), non-Hodgkin’s lymphoma, multiple myeloma, melanoma, hepatocellular carcinoma, head and neck cancer, gastric cancer, endometrial cancer, colorectal cancer, cholangiocarcinoma, breast cancer, bladder cancer, myeloid leukemia, acute lymphoblastic leukemia, acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, esophageal cancer, cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer.

In accordance with the present invention, in one embodiment, prevention and/or treatment of cancer may comprise:
providing one or more of
   (i) the TCR as described and provided herein,
   (ii) the nucleic acid molecule as described and provided herein,
   (iii) the vector as described and provided herein,
   (iv) the host cell as described and provided herein, and
   (v) the pharmaceutical composition as described and provided herein; and administering at least one of (i) to (v) to a subject in need thereof.

In accordance with the present invention, in another embodiment, prevention and/or treatment of cancer may comprise:
(1) providing a sample of a subject, said sample comprising lymphocytes;
(2) providing one or more of
   (i) the TCR as described and provided herein,
   (ii) the nucleic acid molecule as described and provided herein,
   (iii) the vector as described and provided herein,
   (iv) the host cell as described and provided herein, and
   (v) the pharmaceutical composition as described and provided herein;
(3) introducing of one or more of (i) to (v) of step (2) into the lymphocytes of step (1) and, thereby, obtaining modified lymphocytes; and
(4) administering the modified lymphocytes of step (3) to a subject or patient in need thereof.

The present invention further relates to a method of detecting the presence of a cancer in a subject in vitro, comprising:
providing a sample of a subject, said sample comprising one or more cells; contacting said sample with
   (i) the TCR as described and provided herein,
   (ii) the host cell as described and provided herein, and/or
   (iii) the pharmaceutical composition as described and provided herein,
thereby forming a complex; and
detecting the complex,
wherein detection of the complex is indicative of the presence of the cancer in the subject.

The present invention further relates to the use of a TCR as described and provided herein, a nucleic acid molecule as described and provided herein, and/or a vector as described and provided herein, for generating modified lymphocytes.

TABLE 1

Sequences

| | amino acid | nucleic acid |
|---|---|---|
| | SEQ ID NO: | SEQ ID NO: |
| PRAME peptide (PRAME₃₀₁₋₃₀₉) | 2 | 1 |
| T116-49 CDR1 alpha | 4 | 3 |
| T116-49 CDR1 beta | 6 | 5 |
| T116-49 CDR2 alpha | 8 | 7 |
| T116-49 CDR2 beta | 10 | 9 |
| T116-49 CDR3 alpha | 12 | 11 |
| T116-49 CDR3 beta | 14 | 13 |
| T116-49 TCR alpha variable region | 16 | 15 |
| T116-49 TCR beta variable region | 18 | 17 |
| T116-49 TCR alpha chain | 20 | 19 |
| T116-49 TCR beta chain | 22 | 21 |
| TCR alpha murC constant | 24 | 23 |
| TCR beta murC constant | 26 | 25 |
| TCR alpha human constant | 27 | n/a |
| TCR beta human constant | 28 | n/a |
| TCR alpha mm constant | 29 | n/a |
| TCR beta mm constant | 30 | n/a |
| T402-93 CDR1 alpha | 32 | 31 |
| T402-93 CDR1 beta | 34 | 33 |
| T402-93 CDR2 alpha | 36 | 35 |
| T402-93 CDR2 beta | 38 | 37 |
| T402-93 CDR3 alpha | 40 | 39 |
| T402-93 CDR3 beta | 42 | 41 |
| T402-93 TCR alpha variable region | 44 | 43 |
| T402-93 TCR beta variable region | 46 | 45 |
| T402-93 TCR alpha chain (murC) | 48 | 47 |
| T402-93 TCR beta chain (murC) | 50 | 49 |
| PRAME full-length | 51 | n/a |
| Peptide #4 (YYSDSIFFL) | 52 | n/a |
| Peptide #33 (LYVDTIGFL) | 53 | n/a |
| Peptide #38 (DYVDSLYFC) | 54 | n/a |
| Peptide #42 (LYYDHLGFL) | 55 | n/a |
| Peptide #18 (DYVGTLFFL) | 56 | n/a |

The present invention may also be characterized by the following items:

1. A T cell receptor (TCR) capable of binding to
a polypeptide comprising an amino acid sequence according to amino acid sequence LYVDSLFFL (SEQ ID NO: 2) wherein not more than 4 amino acids have been substituted, or
to a portion of said polypeptide, or
to the respective HLA-A bound form of said polypeptide or portion thereof, wherein the TCR comprises:
   (A) a CDR3
      (Aa) of the TCR alpha chain comprising an amino acid sequence being at least 80% similar to SEQ ID NO: 12, and/or
      (Ab) of the TCR beta chain comprising an amino acid sequence being at least 80% similar to SEQ ID NO: 14,
      or
   (B) a CDR3
      (Ba) of the TCR alpha chain comprising an amino acid sequence being at least 80% similar to SEQ ID NO: 40, and/or
      (Bb) of the TCR beta chain comprising of an amino acid sequence being at least 80% similar to SEQ ID NO: 42.

2. The TCR according to item 1,
wherein said TCR comprising a CDR3 according to (A) further comprises
   (Aa1) a CDR1 of the TCR alpha chain comprising an amino acid sequence being at least 80% similar to the amino acid sequence of SEQ ID NO: 4, and/or a CDR2 of the TCR alpha chain comprising an amino acid sequence being at least 80% similar to the amino acid sequence of SEQ ID NO: 8, and/or
   (Ab1) a CDR1 of the TCR beta chain comprising an amino acid sequence being at least 80% similar to the amino acid sequence of SEQ ID NO: 6, and/or a CDR2 of the TCR beta chain comprising an amino acid sequence being at least 80% similar to the amino acid sequence of SEQ ID NO: 10,
or wherein said TCR comprising a CDR3 according to (B) further comprises
   (Ba1) a CDR1 of the TCR alpha chain comprising an amino acid sequence being at least 80% similar to the amino acid sequence of SEQ ID NO: 32, and/or a CDR2 of the TCR alpha chain comprising an amino acid sequence being at least 80% similar to the amino acid sequence of SEQ ID NO: 36, and/or
   (Bb1) a CDR1 of the TCR beta chain comprising an amino acid sequence being at least 80% similar to the amino acid sequence of SEQ ID NO: 34, and/or a CDR2 of the TCR beta chain comprising an amino acid sequence being at least 80% similar to the amino acid sequence of SEQ ID NO: 38.

3. The TCR according to items 1 or 2, wherein the HLA-A is an HLA-A*24, or HLA-A*02 encoded molecule.

4. The TCR according to any one of the preceding items, wherein binding of said TCR to said polypeptide, or a portion thereof, or its HLA-A bound form, induces IFN-gamma secretion by cells comprising said TCR.

5. The TCR according to item 4, wherein said induction of IFN-gamma secretion of cells comprising said TCR is at least 5-fold higher compared to control cells not comprising said TCR upon binding to a polypeptide comprising an amino acid sequence according to amino acid sequence LYVDSLFFL (SEQ ID NO: 2) wherein not more than 4 amino acids have been substituted, or to a portion of said polypeptide, or to the respective HLA-A bound form of said polypeptide or portion thereof.

6. The TCR according to any of the preceding items,
wherein said TCR comprising a CDR3 according to (A) comprises
   (Aa2) a TCR alpha chain variable region
      comprising an amino acid sequence being at least 80% similar to SEQ ID NO: 16, and
      comprising an amino acid sequence being at least 80% similar to positions 47 to 51 of SEQ ID NO: 16, and
      comprising an amino acid sequence being at least 80% similar to positions 69 to 75 of SEQ ID NO: 16, and
      comprising an amino acid sequence being at least 80% similar to positions 109 to 123 of SEQ ID NO: 16,
      and/or
   (Ab2) a TCR beta chain variable region
      comprising an amino acid sequence being at least 80% similar to SEQ ID NO: 18, and
      comprising an amino acid sequence being at least 80% similar to positions 46 to 50 of SEQ ID NO: 18, and
      comprising an amino acid sequence being at least 80% similar to positions 68 to 73 of SEQ ID NO: 18, and
      comprising an amino acid sequence being at least 80% similar to positions 110 to 122 of SEQ ID NO: 18,
   or
wherein said TCR comprising a CDR3 according to (B) comprises
   (Ba2) a TCR alpha chain variable region
      comprising an amino acid sequence being at least 80% similar to SEQ ID NO: 44, and
      comprising an amino acid sequence being at least 80% similar to positions 45 to 49 of SEQ ID NO: 44, and
      comprising an amino acid sequence being at least 80% similar to positions 67 to 73 of SEQ ID NO: 44, and
      comprising an amino acid sequence being at least 80% similar to positions 107 to 121 of SEQ ID NO: 44,
      and/or
   (Bb2) a TCR beta chain variable region
      comprising the amino acid sequence being at least 80% similar to SEQ ID NO: 46, and
      comprising an amino acid sequence being at least 80% similar to positions 44 to 49 of SEQ ID NO: 46, and
      comprising an amino acid sequence being at least 80% similar to positions 67 to 71 of SEQ ID NO: 46, and
      comprising an amino acid sequence being at least 80% similar to positions 108 to 122 of SEQ ID NO: 46.

7. The TCR according to any of the preceding items, further comprising
(i) a TCR alpha chain constant region, and/or
(ii) a TCR beta chain constant region.

8. The TCR according to any of the preceding items,
wherein said TCR comprising a CDR3 according to (A) comprises
   (Aa3) a TCR alpha chain
      comprising an amino acid sequence being at least 80% similar to SEQ ID NO: 20, and
      comprising an amino acid sequence being at least 80% similar to positions 47 to 51 of SEQ ID NO: 20, and
      comprising an amino acid sequence being at least 80% similar to positions 69 to 75 of SEQ ID NO: 20, and
      comprising an amino acid sequence being at least 80% similar to positions 109 to 123 of SEQ ID NO: 20,
      and/or
   (Ab3) a TCR beta chain
      comprising an amino acid sequence being at least 80% similar to SEQ ID NO: 22, and
      comprising an amino acid sequence being at least 80% similar to positions 46 to 50 of SEQ ID NO: 22, and
      comprising an amino acid sequence being at least 80% similar to positions 68 to 73 of SEQ ID NO: 22, and
      comprising an amino acid sequence being at least 80% similar to positions 110 to 122 of SEQ ID NO: 22,
   or
wherein said TCR comprising a CDR3 according to (B) comprises
   (Ba3) a TCR alpha chain
      comprising an amino acid sequence being at least 80% similar to SEQ ID NO: 48, and
      comprising an amino acid sequence being at least 80% similar to positions 45 to 49 of SEQ ID NO: 48, and
      comprising an amino acid sequence being at least 80% similar to positions 67 to 73 of SEQ ID NO: 48, and
      comprising an amino acid sequence being at least 80% similar to positions 107 to 121 of SEQ ID NO: 48,
      and/or
   (Bb3) a TCR beta chain
      comprising an amino acid sequence being at least 80% similar to SEQ ID NO: 50, and
      comprising an amino acid sequence being at least 80% similar to positions 44 to 49 of SEQ ID NO: 50, and
      comprising an amino acid sequence being at least 80% similar to positions 67 to 71 of SEQ ID NO: 50, and
      comprising an amino acid sequence being at least 80% similar to positions 108 to 122 of SEQ ID NO: 50.

9. The TCR according to any of the preceding items, comprising
(A) at least one TCR alpha chain or subregion thereof according to (Aa), (Aa1), (Aa2) or (Aa3), and
   at least one TCR beta chain or subregion thereof according to (Ab), (Ab1), (Ab2) or (Ab3),
   covalently linked to each other to form a TCR heterodimer or multimer,
   or
(B) at least one TCR alpha chain or subregion thereof according to (Ba), (Ba1), (Ba2) or (Ba3), and
   at least one TCR beta chain or subregion thereof according to (Bb), (Bb1), (Bb2) or (Bb3), covalently linked to each other to form a TCR heterodimer or multimer.

10. The TCR according to any one of the preceding items, said TCR being selected from the group consisting of a native TCR, a TCR variant, a TCR fragment, and a TCR construct.

11. The TCR according to any one of the preceding items which is water soluble.

12. The TCR according to any one of the preceding item, further comprising at least one molecular marker.

13. A nucleic acid molecule encoding the TCR according to any one of the preceding items.

14. The nucleic acid according to item 13, comprising the nucleic acid sequence being at least 80% identical to the nucleic acid sequence of any one of SEQ ID NOs:, 3, 5, 7, 9, 11, 13, 15, 17, 19, or 21; or being at least 80% identical to the nucleic acid sequence of any one of SEQ ID NOs: 31, 33, 35, 37, 39, 41, 43, 45, 47, or 49.

15. A vector comprising the nucleic acid molecule according to items 13 or 14.

16. A host cell comprising the TCR according to any one of items 1 to 12, the nucleic acid molecule according to item 13 or 14 or the vector according to item 15.

17. The host cell of item 16 which is selected from lymphocytes including but not limited to lymphoblastoid cell lines, cytotoxic T lymphocytes (CTLs), CD8+ T cells, CD4+ T cells, T memory stem cells (T_{SCM}), natural killer (NK) cells, natural killer T (NKT) cells, and gamma/ delta-T cells.

18. A method for obtaining a TCR according to any of the preceding items, comprising incubating a host cell according to item 16 or 17 under conditions causing expression of said TCR, and purifying said TCR.

19. A pharmaceutical or diagnostic composition comprising one or more of:
(i) the TCR according to any one of items 1 to 12;
(ii) the nucleic acid molecule according to item 13 or 14;
(iii) the vector according to item 15; and/or
(iv) the host cell according to item 16 or 17,
and, optionally, pharmaceutically excipient(s).

20. The pharmaceutical composition according to item 19, further comprising a checkpoint inhibitor.

21. The pharmaceutical composition according to item 20, wherein said checkpoint inhibitor is selected from the group consisting of a CTLA-4 inhibitor, a PD-1 inhibitor and a PD-L1 inhibitor.

22. The TCR according to any one of items 1 to 12, the nucleic acid molecule according to item 13 or 14, the vector according to item 15 and/or the host cell according to item 16 or 17 for use as a medicament.

23. The TCR according to any one of items 1 to 12, the nucleic acid molecule according to item 13 or 14, the vector according to item 15 and/or the host cell according to item 16 or 17 for use in detection, diagnosis, prognosis, prevention and/or treatment of cancer.

24. The TCR, the nucleic acid molecule, the vector or the host cell according to item 23, wherein the cancer is selected from the group consisting of melanoma, bladder carcinoma, colon carcinoma, breast adenocarcinoma, sarcoma, prostate cancer, uterine cancer, uveal cancer, uveal melanoma, squamous head and neck cancer, synovial carcinoma, Ewing’s sarcoma, triple negative breast cancer, thyroid cancer, testicular cancer, renal cancer, pancreatic cancer, ovarian cancer, esophageal cancer, non-small-cell lung cancer (NSCLC), small-cell lung cancer (SCLC), non-Hodgkin’s lymphoma, multiple myeloma, melanoma, hepatocellular carcinoma, head and neck cancer, gastric cancer, endometrial cancer, colorectal cancer, cholangiocarcinoma, breast cancer, bladder cancer, myeloid leukemia, acute lymphoblastic leukemia, acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, esophageal cancer, cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer.

25. The TCR, nucleic acid, vector and/or host cell for the use of item 23 or 24, wherein prevention and/or treatment of cancer comprises:
providing one or more of
   (i) the TCR according to any one of items 1 to 12,
   (ii) the nucleic acid molecule according to item 13 or 14,
   (iii) the vector according to item 15,
   (iv) the host cell according to item 16 or 17, and
   (v) the pharmaceutical composition of any one of items 19 to 21; and administering at least one of (i) to (v) to a subject in need thereof.

26. The TCR according to any one of items 1 to 12, the nucleic acid molecule according to item 13 or 14, the vector according to item 15 and/or the host cell according to item 16 or 17 for use of any one of items 23 to 25, wherein prevention and/or treatment of cancer comprises:
(1)providing a sample of a subject, said sample comprising lymphocytes;
(2) providing one or more of
   (i) the TCR according to any one of items 1 to 12,
   (ii) the nucleic acid molecule according to item 13 or 14,
   (iii) the vector according to item 15,
   (iv) the host cell according to item 16 or 17, and
   (v) the pharmaceutical composition of any one of items 19 to 21;
(3) introducing of one or more of (i) to (v) of step (2) into the lymphocytes of step (1) and, thereby, obtaining modified lymphocytes; and
(4) administering the modified lymphocytes of step (3) to a subject or patient in need thereof.

27. A method of detecting the presence of a cancer in a subject in vitro, comprising: providing a sample of a subject, said sample comprising one or more cells; contacting said sample with
(i) the TCR according to any one of items 1 to 12,
(ii) the host cell according to item 16 or 17, and/or
(iii) the pharmaceutical composition of any one of items 19 to 21, thereby forming a complex; and
detecting the complex, wherein detection of the complex is indicative of the presence of the cancer in the subject.

28. Use of a TCR according to any one of items 1 to 12, a nucleic acid molecule according to item 13 or 14, and/or a vector according to item 15, for generating modified lymphocytes.

The embodiments which characterize the present invention are described herein, shown in the Figures, illustrated in the Examples, and reflected in the claims.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% or 2% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer’s specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### FIGURES

The Figures show:

FIG. 1 A lymphoblastoid cell line (LCL; EBV-transformed B cells) expressing HLA-A*24:02-encoded molecules was electroporated with either ivtRNA encoding PRAME or water and loaded with either PRAME₃₀₁₋₃₀₉ peptide or with an irrelevant peptide. These cells were used as targets in a co-culture assay with either TCR T402-93- or TCR T116-49- transgenic T cells. Untransduced T cells served as negative control. After 24 hours (h) of incubation, IFN-γ release by TCR transgenic T cells was assessed by standard ELISA. This experiment was performed with two different donors. Shown is one representative experiment.

FIG. 2 CD8⁺ T cells expressing either TCR T402-93 or TCR T116-49 were co-cultured with either HLA-A*24-positive PRAME-positive tumor cell lines (K562, Mel624.38, CMK, SKHEP1) or HLA-A*24-positive PRAME-negative tumor cell lines (Colo678, MCF-7, 22RV1). Untransduced CD8⁺ T cells served as negative control. An HLA-A*24-positive LCL electroporated with either ivtRNA encoding PRAME or water and loaded with either PRAME₃₀₁₋₃₀₉ peptide or with an irrelevant peptide was included as internal target controls. Activation of transgenic TCR-expressing T cells was evaluated after 24h co-culture by standard ELISA measuring IFN-γ release in [pg/ml]. Shown is the mean value of duplicates with standard deviations. This experiment was performed with two different donors. Shown is one representative experiment. Values above 4000 pg were extrapolated using a third-degree polynomial.

FIG. 3 Red-labelled tumor cell lines were incubated with either TCR T402-93- or TCR T116-49-transgenic T cells and with untransduced T cells. The cells were monitored using a live-cell imaging system over a period of 105h to evaluate the killing of red-labelled tumor cells mediated by TCR-transgenic T cells. The total integrated intensity (RCU (red calibrated unit) x µm²/image) was calculated using the IncuCyte ZOOM® software. Each measurement point represents the mean of three technical replicates. This experiment was performed with two different donors. Shown is one representative experiment.

FIG. 4 An HLA-A*24-positive LCL was loaded with titrated amounts (10⁻⁵ M to 10⁻⁹ M) of PRAME₃₀₁₋₃₀₉ peptide and co-cultured with either TCR T402-93- or TCR T116-49-expressing CD8⁺ T cells. A standard ELISA was performed after 24h to evaluate IFN-γ release by T cells. Maximal IFN-γ release per effector cell sample was set to 100%. Based on this, the relative IFN-γ release was calculated. This experiment was performed with two different donors. Shown is one representative experiment.

FIG. 5 Threonine scanning assay was performed for the 9-mer PRAME₃₀₁₋₃₀₉ (LYVDSLFFL) peptide recognized by TCR T402-93 and TCR T116-49. The amino acids included in PRAME₃₀₁₋₃₀₉ peptide were consecutively replaced by a threonine (exchanged aa are shown in bold). An HLA-A*24-positive LCL was loaded with the modified peptides (10⁻⁵M) and used in co-culture with T cells expressing either TCR T402-93 or TCR T116-49 and with untransduced T cells to evaluate IFN-γ secretion by standard ELISA after 24 h of incubation. Shown is the mean value of duplicates with standard deviations. This experiment was performed with two different donors. Shown is one representative experiment.

FIG. 6 Peptides with up to three amino acid differences compared to wild-type 9-mer PRAME₃₀₁₋₃₀₉ (LYVDSLFFL) peptide were selected using Expitope 2.0® tool. Mismatched peptides were loaded on HLA-A*24-positive LCL (10⁻⁵M) and recognition by either TCR T402-93- or TCR T116-49-transgenic T cells was tested. PRAME₃₀₁₋₃₀₉-loaded LCL were included as internal positive control. Activation of T cells was assessed using standard ELISA IFN-γ after 24h of incubation. This experiment was performed with two different donors. Shown is one representative experiment.

FIG. 7 TCR T116-49-transduced T cells were co-cultured with a cellular library consisting of 52 LCLs covering the most frequent HLA-A, -B and —C alleles in the German and USA/European Caucasian populations. In addition, the same 52 LCLs were loaded with PRAME₃₀₁₋₃₀₉ peptide and tested in co-culture with TCR T116-49-transduced T cells. A standard ELISA was performed after 24h of incubation to evaluate IFN-γ release by T cells. This experiment was performed with two different donors. Shown is one representative experiment.

The present invention is further illustrated by the following examples. Yet, the examples and specific embodiments described therein must not be construed as limiting the invention to such specific embodiments.

### EXAMPLES

### Example 1: Isolation of PRAME₃₀₁₋₃₀₉-specific HLA-A24-restricted TCRs

An in vitro priming approach was used to isolate T cell clones of any desired HLA restriction and antigen specificity. The priming system used mature dendritic cells (mDCs) of an HLA-A*24:02-negative healthy donor as antigen-presenting cells and autologous CD8-enriched T cells as responding cells. In vitro transcribed RNA (ivtRNA) encoding the full-length human PRAME amino acid sequence served as the source of specific antigen. Simultaneously, human HLA-A*24:02-encoding ivtRNA (sequence derived from https://www.ebi.ac.uk/ipd/imgt/hla/) was used as source of restriction element and transfected into mDCs to set-up an allogeneic priming in terms of this dedicated HLA allele (as described in WO2007/017201). After electroporation into the mDCs, the PRAME-encoding ivtRNA was translated into full-length protein, which was subsequently processed and presented as peptides by transgenic HLA-A*24 molecules which are expressed by transfected mDCs. In vitro co-cultures of T cells with ivtRNA-transfected mDCs from the same donor led to de novo induction of antigen-specific T cells that served as the source of corresponding TCRs.

Allogeneic T cell priming approach using mDCs transfected with HLA-A*24:02-encoding ivtRNA and with PRAME ivtRNA was accomplished using peptide presentation by allogeneic HLA-A*24:02-encoded molecules according to the following protocol:

### HLA-A*24:02/PRAME Priming

Monocytes were derived from HLA-A*24:02-negative healthy donors and corresponding mDCs were produced using a suitable maturation cocktail according to Jonuleit et al. protocol (Jonuleit et al., Eur. J. Immunol. 1997, 27:3135-3142). mDCs were electroporated simultaneously with 20 µg ivtRNA encoding for PRAME and 20 µg ivtRNA encoding HLA-A*24 molecule. The prepared mDCs were subsequently co-cultured with autologous CD8⁺ T cells in a ratio of 1:10 for about 14 days in a suitable cell medium supplemented with IL-2 (50 units/ml). Subsequently, PRAME₃₀₁₋₃₀₉-specific T cells were identified using HLA-A*24:02 PRAME₃₀₁₋₃₀₉ multimer and separated by single cell sorting using FACS technology. Following the identification of promising T cells clones that recognized the desired PRAME₃₀₁₋₃₀₀ epitope on HLA-A*24 molecules, the corresponding T cell receptor (TCR) sequences were analyzed by next-generation sequencing (NGS). The identified HLA-A*24-restricted PRAME₃₀₁₋₃₀₉ -specific TCRs (T402-93 and T116-49) were expressed into recipient T cells and a characterization regarding function and specificity was conducted.

### Example 2: Evaluation of Antigen Specificity

An LCL expressing HLA-A*24:02-encoded molecules was loaded with either the specific PRAME₃₀₁₋₃₀₉ peptide or an irrelevant peptide at a concentration of 10⁻⁵ M. Additionally, the same HLA-A*24-positive LCL was electroporated with either ivtRNA encoding PRAME or water as negative control. Each target cell line was co-cultured with T cells transduced with either TCR T402-93 or TCR T116-49 at an effector to target (E:T) ratio of 1:2 using 10000 T cells and 20000 targets/96-well. Untransduced T cells (UT) were included as negative control. After 24h of co-culture, IFN-γ released by T cells was measured by standard ELISA.

### Results:

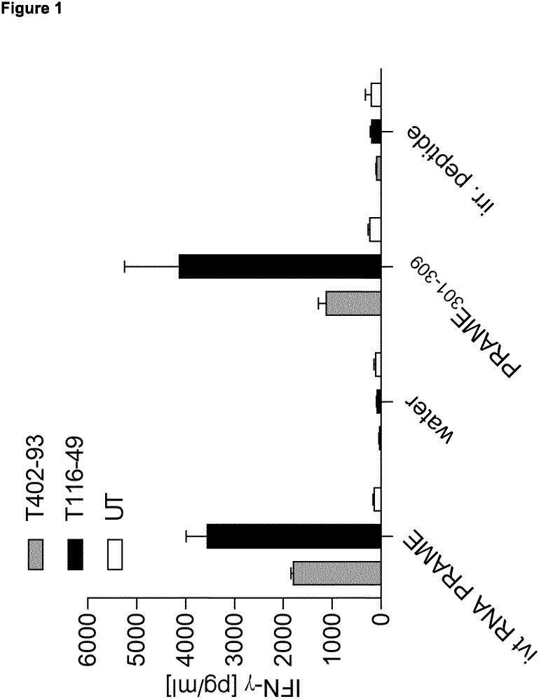

Both TCR T402-93- and TCR T116-49 -transduced T cells recognized the specific PRAME₃₀₁₋₃₀₉ peptide as well as PRAME-transfected LCL. To note, T cells expressing TCR T116-49 showed higher level of released IFN-γ after incubation with positive targets compared to TCR T402-93-transgenic T cells. No recognition of LCL loaded with irrelevant peptide and water-electroporated was observed (FIG. 1).

### Example 3: Tumor Cell Recognition

### Evaluation of IFN-γ Release by T Cells

Effector T cells transduced with either TCR T402-93 or TCR T116-49 were co-cultured with either PRAME-positive tumor cell lines (K562, Mel624.38, CMK, SKHEP1) or PRAME-negative tumor cell lines (Colo678, MCF-7, 22RV1). Of the selected tumor cell lines, CMK and SKHEP1 cell lines are endogenously positive for HLA-A*24, while the other five cell lines are endogenously HLA-A*24-negative. Thus, these five cell lines were tested after either transduction with HLA-A*24 (K562, Mel624.38, 22RV1) or transfection with iviRNA encoding HLA-A*24 molecule (Colo678 and MCF-7). Untransduced CD8⁺ T cells served as negative control. An HLA-A*24-positive LCL electroporated with either ivtRNA encoding PRAME or water and loaded with either PRAME₃₀₁₋₃₀₉ peptide or with an irrelevant peptide was included as internal control. T cells and target cells were co-cultured at an E:T ratio of 1:1 (10000 E/10000 T/96-well). Activation of transgenic TCR-expressing T cells was evaluated after 24 h co-culture by standard ELISA measuring IFN-γ release in [pg/ml]. Values above 4000 pg were extrapolated using a third-degree polynomial.

### Results:

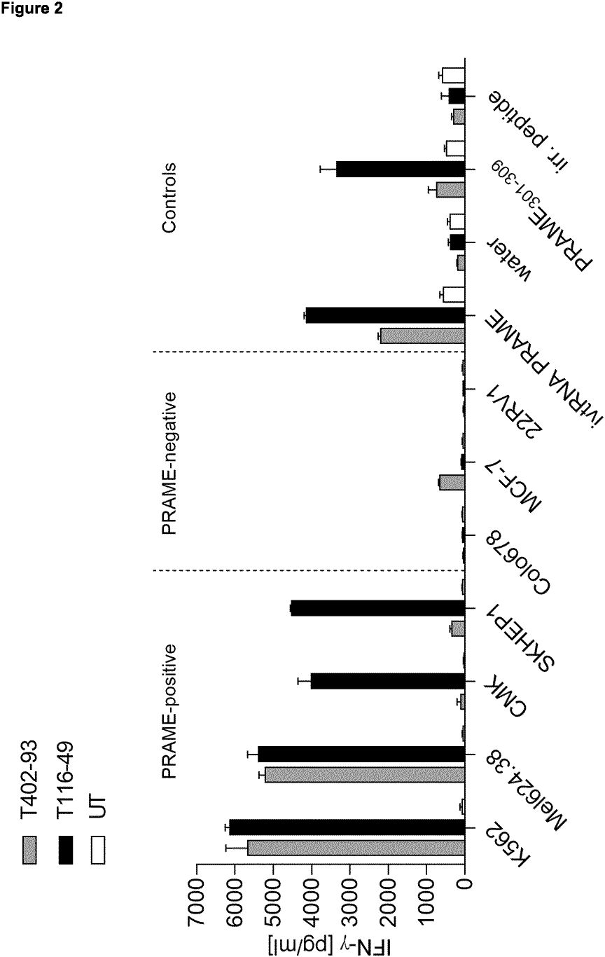

TCR T116-49-transduced T cells showed recognition of all tested PRAME-positive tumor cells, while TCR T402-93-transduced T cells released high level of IFN-γ only after co-culture with two out of four PRAME-positive cells (K562 and Mel624.38) . No recognition of any PRAME-negative cells was observed in co-culture with TCR T116-49-transduced T cells. In contrast, a slight recognition of a PRAME-negative cell line (MCF-7) was observed for T cells expressing TCR 402-93 (FIG. 2).

### Evaluation of Killing Mediated by T Cells

To assess killing mediated by TCR transgenic T cells, two PRAME-positive tumor cell lines (Mel624.38, SKHEP1) and a PRAME-negative tumor cell line (Colo678) were selected as target cells. Of the selected tumor cell lines, SKHEP1 cell line is endogenously positive for HLA-A*24, while the other three cell lines are endogenously HLA-A*24-negative. Thus, SKHEP1 cells were transduced with only mCherry (red fluorescent protein), while the other two cell lines were transduced with HLA-A*24 linked to mCherry. Red-labelled tumor cells were seeded in 96-well flat-bottom plate two days prior to the start of the co-culture (Mel624.38 and SKHEP1 5000 cells/well, while Colo678 10000 cells/well). As internal positive control, the same tumor cell lines were additionally loaded with PRAME₃₀₁₋₃₀₉ peptide. After adding 10000 T cells expressing either TCR T402-93- or TCR T116-49 per well, the co-culture plates were transferred to a live-cell imaging system (IncuCyte ZOOM® device). The cells were monitored over a total period of 105h to assess the killing of red-labelled tumor cells mediated by TCR-transgenic T cells. The total sum of the objects’ red fluorescent intensity in the image, designated total integrated intensity (RCU (red calibrated unit) x µm²/image), was calculated using the IncuCyte ZOOM® software.

### Results:

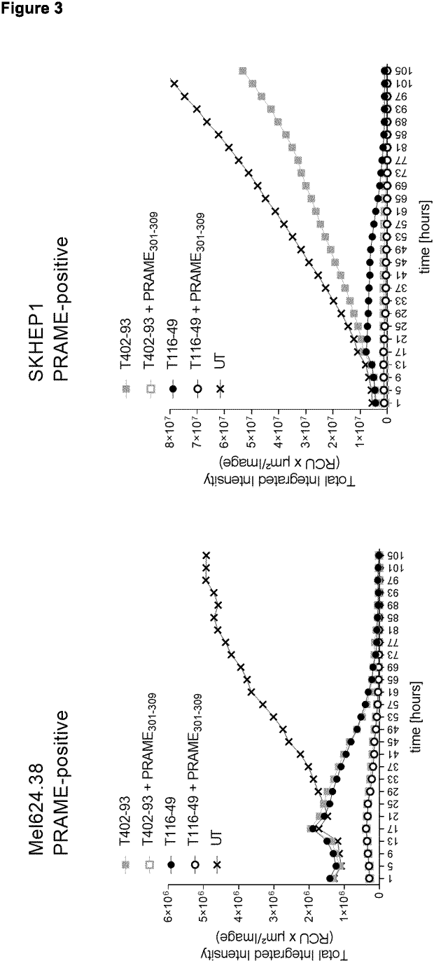

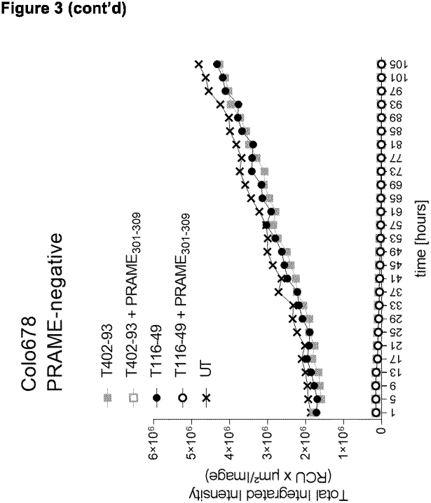

Both TCR-transduced samples affected the growth of Mel624.38 PRAME-positive cell line, in contrast only TCR T116-49-transduced T cells mediated efficient killing of SKHEP1 PRAME-positive cell line. Both TCR-transduced samples did not influence the expansion of PRAME-negative tumor cell. Each target cell line after peptide loading was efficiently killed by TCR-transduced T cells. In contrast, growing target cells were observed for all tumor cell lines when untransduced T cells were used as effectors in the co-culture (FIG. 3).

### Example 4: Functional Avidity

The aim of the experiment was to measure functional avidity of PRAME₃₀₁₋₃₀₉ -specific TCRs. Functional avidity refers to the accumulated strength of multiple affinities of individual noncovalent binding interactions, such as between the transgenic TCR and the pMHC complex. Functional avidities of TCR-transgenic T cell populations were measured as the half-maximal relative IFN-γ release in co-culture with HLA-A*24-positive LCL loaded with titrated amounts of PRAME₃₀₁₋₃₀₉ peptide (10⁻⁵ M to 10⁻⁹ M). T cells and target cells were co-cultured at an E:T ratio of 1:1 (10000 E/10000 T/96-well). Untransduced CD8⁺ T cells were used as internal control for subtracting the reactivity mediated by endogenous TCRs of the T cells and not related to transgenic TCR-specific recognition. A standard ELISA was performed after 24 h to evaluate IFN-γ release by T cells. Maximal IFN-γ release per effector cell sample was set to 100%. Based on this, the relative IFN-γ release was calculated. This experiment was performed with two different donors. Shown is one representative experiment.

### Results:

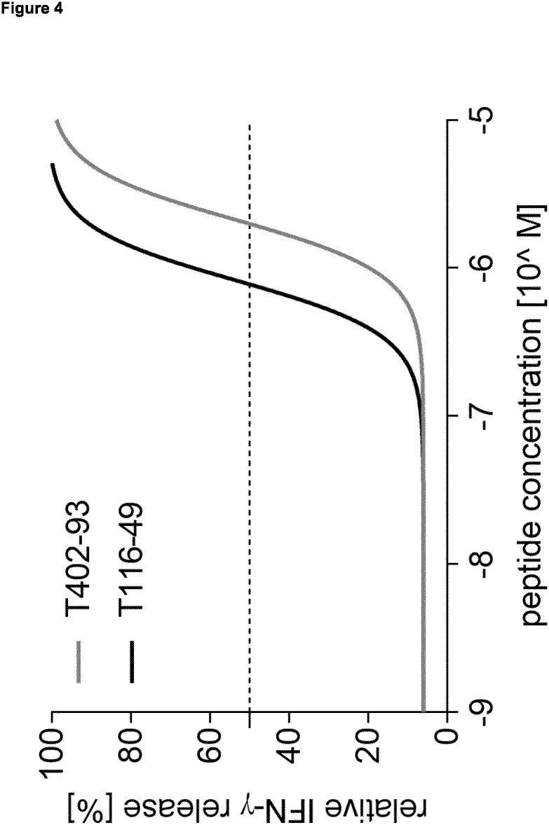

TCR T116-49-transduced T cells showed a higher functional avidity compared to TCR T402-93-transduced T cells, indicating a higher sensitivity for the target peptide (FIG. 4).

### Example 5: TCR Recognition Motif (Threonine Scan Assay)

The aim of the experiment was to assess critical residues within the PRAME₃₀₁₋₃₀₉ epitope that are either essential for direct recognition by the TCR or for peptide binding to the HLA-A*24:02- encoded molecule. Amino acid substitution scanning was used to define critical amino acids in the epitope sequence that abolish recognition by the TCR whenever these residues are exchanged for the amino acid threonine. These “fixed” amino acids can be used to define unique TCR recognition motifs. Threonine scanning assay was performed for the 9-mer PRAME₃₀₁₋₃₀₉ peptide recognized by TCR T402-93 and TCR T116-49. The amino acids included in PRAME₃₀₁₋₃₀₉ peptide were consecutively replaced by a threonine. An HLA-A*24-positive LCL was loaded with the modified peptides (10⁻⁵M) as well as with the wild-type PRAME₃₀₁₋₃₀₉ peptide and used in co-culture with T cells expressing either TCR T402-93 or TCR T116-49. Untransduced T cells served as internal control. T cells and target cells were co-cultured at an E:T ratio of 1:1 (10000 E/10000 T/96-well). To evaluate IFN-γ secretion by T cells a standard ELISA was performed after 24h of co-culture.

### Results:

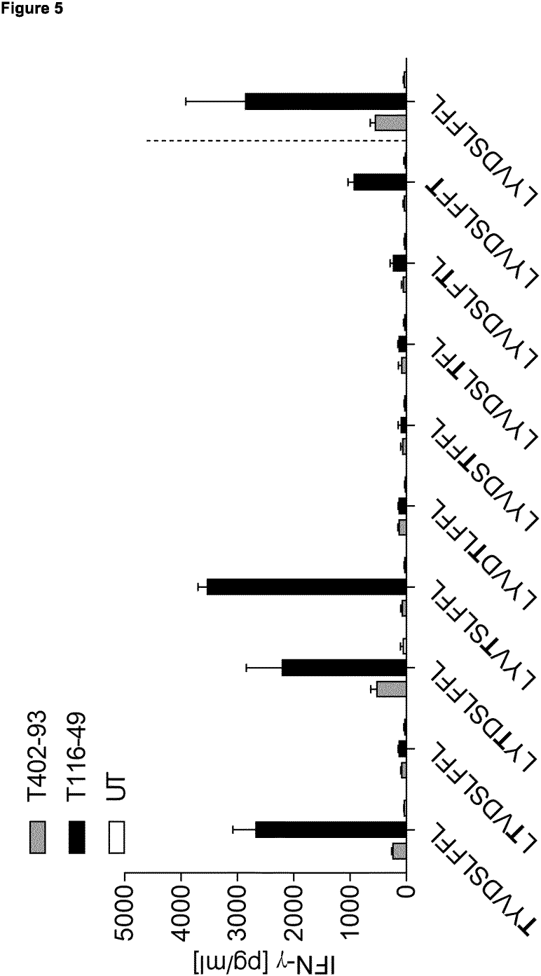

TCR T116-49-transduced T cells showed a different TCR recognition motif with less fixed positions compared to TCR T402-93-transduced T cells (FIG. 5).

### Example 6: Recognition of Mismatched Peptides

By in silico analysis using Expitope 2.0® tool (Expitope® 2.0; Jaravine et al. BMC Cancer 2017), 52 peptides including up to 3 mismatches compared to the wild-type 9-mer PRAME₃₀₁₋₃₀₉ epitope were selected. Mismatched peptides were loaded on HLA-A*24-positive LCL (10⁻ ⁵M) and recognition by TCR T402-93- and TCR T116-49-transgenic T cells was tested. Wild-type PRAME₃₀₁₋₃₀₉ peptide-loaded LCL as well as unloaded LCL were included as internal controls. T cells and target cells were co-cultured at an E:T ratio of 1:1 (10000 E/10000 T/96-well). Activation of T cells was assessed using standard ELISA IFN-γ after 24h of incubation

### Results:

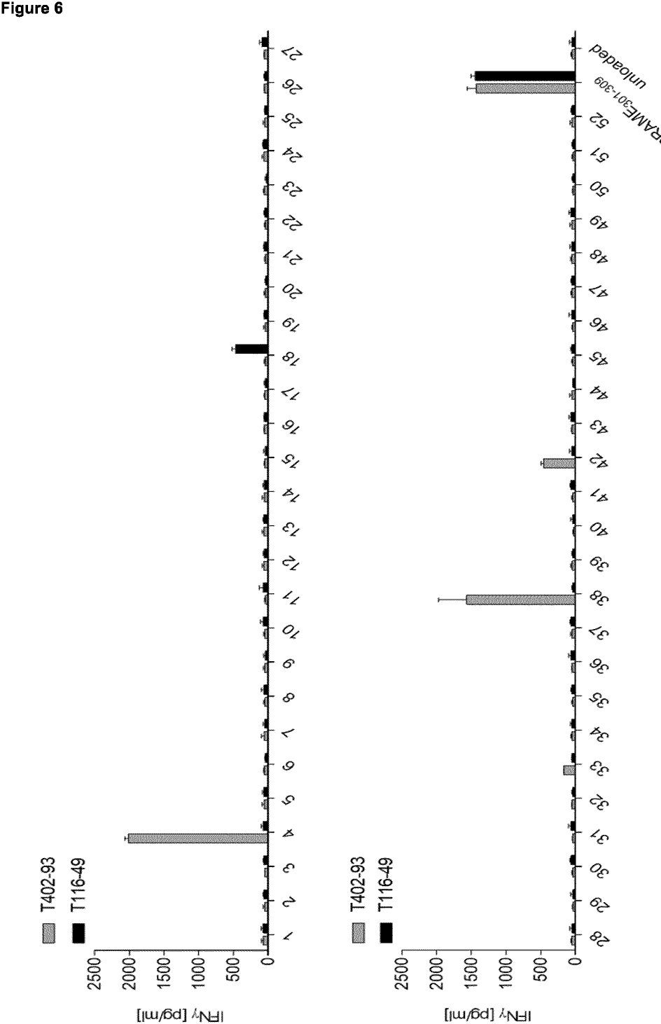

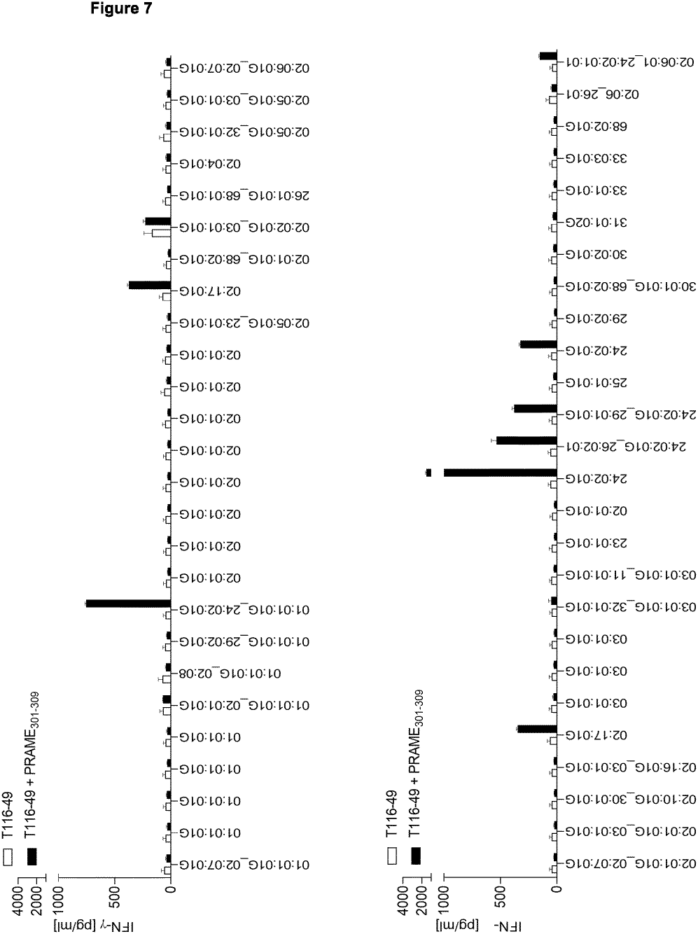

TCR transgenic T cell samples recognized the wild-type PRAME₃₀₁₋₃₀₉ peptide but not unloaded targets and therefore proved functionality of the transgenic T cells. TCR T402-93-transduced T cells were activated also by target cells loaded with peptide #4, #33, #38 and #42, while TCR T116-49-transduced T cells release IFN-γ upon stimulation with LCL loaded with the mismatched peptide #18 (FIG. 6 and Table 2).

TABLE 2

List of the five mismatched peptides out of 52 tested peptides recognized by either T402-93- or TCR T116-49-transgenic T cells wild-type PRAME₃₀₁₋₃₀₉ peptide: LYVDSLFFL mm peptide recognized by T402-93

| # | Peptide | numbers of mismatches | Gene |
|---|---|---|---|
| 4 | YYSDSIFFL | 3 | XXYLT1 |
| 33 | LYVDTIGFL | 3 | GTPBP6 |
| 38 | DYVDSLYFC | 3 | KCNK12 |
| 42 | LYYDHLGFL | 3 | CATSPERB |

mm peptide recognized by T116-49

| # | Peptide | numbers of mismatches | Gene |
|---|---|---|---|
| 18 | DYVGTLFFL | 3 | FBXO24 |

Peptide #4 (YYSDSIFFL) is shown in SEQ ID NO: 52, peptide #33 (LYVDTIGFL) is shown in SEQ ID NO: 53, peptide #38 (DYVDSLYFC) is shown in SEQ ID NO: 54, peptide #42 (LYYDHLGFL) is shown in SEQ ID NO: 55 and peptide #18 (DYVGTLFFL) is shown in SEQ ID NO: 56.

### Example 7: LCL Library

A cellular library consisting of 52 LCLs covering the most frequent HLA-A, -B and -C alleles in the German and USA/European Caucasian populations was established. HLA allele frequencies of more than 0.5% in either of these populations were covered by at least one cell line, HLA alleles exhibiting frequencies over 5% are covered by at least two LCLs (except HLA-A*11:01). First aim of the experiment was to investigate potential target antigen-independent cross-recognition of frequent HLA allotypes by the TCR 116-49. HLA-allo cross-recognition can be defined as the ability of the TCR to interact with allogeneic HLA molecules whereby these interactions are also described to exhibit exquisite peptide and HLA specificities. Thus, the 52 LCLs were incubated with T cells expressing TCR T116-49. Additional aim of the experiment was to determine common HLA-A sub-alleles other than HLA-A*24:02 that are able to present the PRAME₃₀₁₋₃₀₉ epitope and can be recognized by the TCR T116-49-transgenic T cells (HLA restriction fine-typing). Therefore, the 52 LCLs were loaded with PRAME₃₀₁₋₃₀₉ peptide (10⁻⁵M) and subsequently used as targets in a co-culture with TCR T116-49-transgenic T cells. After 24h of incubation, a standard ELISA was performed to measure IFN-γ release by T cells.

### Results:

IFN-γ release by TCR 116-49-transduced T cells was observed after co-culture with all PRAME₃₀₁₋₃₀₉ peptide-loaded HLA-A*24:02-positive LCLs included in the library. TCR 116-49-transgenic T cells slightly recognized LCL expressing HLA-A*02:02 without peptide loading as well as after loading of PRAME₃₀₁₋₃₀₉ peptide, suggesting potential HLA-allo cross recognition for HLA-A*02:02 allele. The two LCLs expressing HLA-A*02:17 were recognized by TCR T116-49-transgenic T cells only after PRAME₃₀₁₋₃₀₉ peptide loading, showing that PRAME₃₀₁₋₃₀₉ epitope might also be presented on HLA-A*02:17-encoded molecule leading to activation of TCR T116-49-transgenic T cells.

Sequences (in case of conflict the following sequences rule over the sequences of the sequence listing according to WIPO ST.25 standard):

| SEQ ID NO. | |
|---|---|
| 1 | ctctatgtggactctttatttttcctt |
| 2 | LYVDSLFFL |
| 3 | aaggccctgtacagc |
| 4 | KALYS |
| 5 | gagaaccatcggtac |
| 6 | ENHRY |
| 7 | ctgctgaaaggcggcgagcag |
| 8 | LLKGGEQ |
| 9 | agctacggcgtgaaggac |
| 10 | SYGVKD |
| 11 | tgcggcacagccaatagcggcggcagcaactacaagctgaccttc |
| 12 | CGTANSGGSNYKLTF |
| 13 | tgcgccatcagcgactacgagggcaccgaggcctttttt |
| 14 | CAISDYEGTEAFF |
| 15 | atggagacactgctgaaggtgctgtctggcacactgctgtggcagctgacctgggtccgatctcagcagcctgttcagtctcctcaggccgtgatcctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgcggcacagccaatagcggcggcagcaactacaagctgacottcggcaagggcaccctgctgaccgtgaatcccaat |
| 16 | METLLKVLSGTLLWQLTWVRSQQPVQSPQAVILREGEDAVINCSSSKALYSVHWYRQKH GEAPVFLMILLKGGEQKGHEKISASFNEKKQQSSLYLTASQLSYSGTYFCGTANSGGSNY KLTFGKGTLLTVNPN |
| 17 | atgggcaccagactgttcttctacgtggccctgtgtctgctgtggacaggccatgtggatgccggaatcacacagagccccagacacaaagtgaccgagacaggcacccctgtgacactgagatgtcaccagaccgagaaccatcggtacatgtattggtacagacaggaccccggccacggcctgagactgatccactatagctacggcgtgaaggacaccgacaagggcgaagtgtctgacggctacagcgtgtccagaagcaagaccgaggacttcctgctgaccctggaaagcgccacaagcagccagaccagcgtgtacttctgcgccatcagcgactacgagggcaccgaggccttttttggccaaggcacaagactgaccgtggtg |
| 18 | MGTRLFFYVALCLLWTGHVDAGITQSPRHKVTETGTPVTLRCHQTENHRYMYWYRQDPG HGLRLIHYSYGVKDTDKGEVSDGYSVSRSKTEDFLLTLESATSSQTSVYFCAISDYEGTEA FFGQGTRLTVV |
| 19 | atggagacactgctgaaggtgctgtctggcacactgctgtggcagctgacctgggtccgatctcagcagcctgttcagtctcctcaggccgtgatcctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgcggcacagccaatagcggcggcagcaactacaagctgacottcggcaagggcaccctgctgaccgtgaatcccaatatccagaatccggagcccgccgtataccagctgaaggaccctagaagccaggacagcaccctgtgcctgttcaccgacttcgacagccagatcaacgtgcccaagaccatggaaageggceaccttcatcaccgacaagacagtgcetggacatgaaggecatggacagcaagtccaacggcgcaatcgcctggtccaaccagaccagcttcacatgccaggacatcttcaaagagacaaacgccacataccccagcagcgacgtgccctgtgatgccaccctgacagagaagtccttcgagacagacatgaacctgaacttccagaatctgtccgtgatgggcctgagaatcctgctgctgaaggtggccggcttcaatctgctgatgaccctgcggctgtggtccagc |
| 20 | METLLKVLSGTLLWQLTWVRSQQPVQSPQAVILREGEDAVINCSSSKALYSVHWYRQKH GEAPVFLMILLKGGEQKGHEKISASFNEKKQQSSLYLTASQLSYSGTYFCGTANSGGSNY KLTFGKGTLLTVNPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS |
| 21 | atgggcaccagactgttcttctacgtggcecotgtgtctgetgtggacaggcecatgtggatgccggaatcacacagagccccagacacaaagtgaccgagacaggcacccctgtgacactgagatgtcaccagaccgagaaccatcggtacatgtattggtacagacaggaccccggccacggcctgagactgatccactatagctacggcgtgaaggacaccgacaagggcgaagtgtctgacggctacagcgtgtccagaagcaagaccgaggacttcctgctgaccctggaaagcgccacaagcagccagaccagcgtgtacttctgcgccatcagcgactacgagggcaccgaggccttttttggccaaggcacaagactgaccgtggtggaagatctccggaacgtgaccccccctaaagtgaccctgttcgaacccagcaaggccgagatcgccaacaagcagaaagccaccctcgtgtgcctggccagaggcttcttccccgaccatgtggaactgtcttggtgggtcaacggcaaagaggtgcacagcggagtgtccaccgaccctcaggcctacaaagagagcaactacagctactgcctgagcagcagactgcgggtgtccgccaccttctggcacaacccccggaaccacttcagatgccaggtgcagtttcacggcctgagcgaagaggacaagtggcccgaaggctcccccaagcccgtgacccagaatatctctgccgaggcctggggcagagccgactgtggaattaccagcgccagctaccaccagggcgtgctgtctgccaccatcctgtacgagatcctgctgggcaaggccaccctgtacgccgtgctggtgtctggcctggtgctgatggccatggtcaagaagaagaacagc |
| 22 | MGTRLFFYVALCLLWTGHVDAGITQSPRHKVTETGTPVTLRCHQTENHRYMYWYRQDPG HGLRLIHYSYGVKDTDKGEVSDGYSVSRSKTEDFLLTLESATSSQTSVYFCAISDYEGTEA FFGQGTRLTVVEDLRNVTPPKVTLFEPSKAEIANKQKATLVCLARGFFPDHVELSWWVNG KEVHSGVSTDPQAYKESNYSYCLSSRLRVSATFWHNPRNHFRCQVQFHGLSEEDKWPE GSPKPVTQNISAEAWGRADCGITSASYHQGVLSATIL YEILLGKATL YAVLVSGLVLMAMV KKKNS |
| 23 | atccagaatccggagcccgccgtataccagctgaaggaccctagaagccaggacagcaccctgtgcctgttcaccgacttcgacagccagatcaacgtgcccaagaccatggaaagcggcaccticatcaccgacaagacagtgctggacatgaaggcecatggacagcaagtccaacggcgcaatcgcctggtccaaccagaccagcttcacatgccaggacatcttcaaagagacaaacgccacataccccagcagcgacgtgccctgtgatgccaccctgacagagaagtccttcgagacagacatgaacctgaacttccagaatctgtccgtgatgggcctgagaatcctgctgctgaaggtggccggcttcaatctgctgatgaccctgcggctgtggtccagc |
| 24 | IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS |
| 25 | gaagatctccggaacgtgaccccccctaaagtgaccctgttcgaacccagcaaggccgagatcgccaacaagcagaaag ccaccctcgtgtgcctggccagaggcttcttccccgaccatgtggaactgtcttggtgggtcaacggcaaagaggtgcacagc ggagtgtccaccgaccctcaggcctacaaagagagcaactacagctactgcctgagcagcagactgcgggtgtccgccac cttctggcacaacccccggaaccacttcagatgccaggtgcagtttcacggcctgagcgaagaggacaagtggcccgaaggctcccccaagcccgtgacccagaatatctctgccgaggcctggggcagagccgactgtggaattaccagcgccagctaccaccagggcgtgctgtctgccaccatcctgtacgagatcctgctgggcaaggccaccctgtacgccgtgctggtgtctggcctggtgctgatggccatggtcaagaagaagaacagc |
| 26 | EDLRNVTPPKVTLFEPSKAEIANKQKATLVCLARGFFPDHVELSWWVNGKEVHSGVSTDP QAYKESNYSYCLSSRLRVSATFWHNPRNHFRCQVQFHGLSEEDKWPEGSPKPVTQNISA EAWGRADCGITSASYHQGVLSATILYEILLGKATLYAVLVSGLVLMAMVKKKNS |
| 27 | IQNPDPAVYQLRDSKSSDKSVCLFTDFDSQTNVSQSKDSDVYITDKTVLDMRSMDFKSNS AVAWSNKSDFACANAFNNSIIPEDTFFPSPESSCDVKLVEKSFETDTNLNFQNLSVIGFRILLLKVAGFNLLMTLRLWSS |
| 28 | EDLNKVFPPEVAVFEPSEAEISHTQKATLVCLATGFFPDHVELSWWVNGKEVHSGVSTDP QPLKEQPALNDSRYCLSSRLRVSATFWQNPRNHFRCQVQFYGLSENDEWTQDRAKPVT QIVSAEAWGRADCGFTSVSYQQGVLSATILYEILLGKATLYAVLVSALVLMAMVKRKDF |
| 29 | IQNPDPAVYQLRDSKSSDKSVCLFTDFDSQTNVSQSKDSDVYITDKTVLDMRSMDFKSNS AVAWSNKSDFACANAFNNSIIPEDTFFPSSDVPCDVKLVEKSFETDTNLNFQNLSVIGFRILLLKVAGFNLLMTLRLWSS |
| 30 | EDLNKVFPPEVAVFEPSKAEIAHTQKATLVCLATGFFPDHVELSWVWNGKEVHSGVSTDP QPLKEQPALNDSRYCLSSRLRVSATFWQNPRNHFRCQVQFYGLSENDEWTQDRAKPVT QIVSAEAWGRADCGITSASYHQGVLSATILYEILLGKATLYAVLVSALVLMAMVKRKDF |
| 31 | accacactgagcaac |
| 32 | TTLSN |
| 33 | ggcaccagcaatcccaac |
| 34 | GTSNPN |
| 35 | ctggtcaagtccggcgaagtg |
| 36 | LVKSGEV |
| 37 | agcgtcggcatcggc |
| 38 | SVGIG |
| 39 | tgtgctggcgccctgcctagagccggcagctatcaactgacattc |
| 40 | CAGALPRAGSYQLTF |
| 41 | tgtgcttggagcctcggagccggctacaccgacacacagtatttt |
| 42 | CAWSLGAGYTDTQYF |
| 43 | atgctgctgatcacctccatgctggtgctgtggatgcagctgagccaagtgaacggccagcaagtgatgcagatccctcagta ccagcacgtgcaagaaggcgaggacttcaccacctactgcaacagcagcaccacactgagcaacatccagtggtacaag cagcggcctggcggacaccctgtgtttctgatccagctggtcaagtccggcgaagtgaagaagcagaagcggctgaccttccagttcggcgaggccaagaagaacagcagcctgcacatcaccgccacacagaccaccgatgtgggcacctacttttgtgctggcgccctgcctagagccggcagctatcaactgacattcggcaagggcaccaagctgagcgtgatccccaac |
| 44 | MLLITSMLVLWMQLSQVNGQQVMQIPQYQHVQEGEDFTTYCNSSTTLSNIQWYKQRPGG HPVFLIQLVKSGEVKKQKRLTFQFGEAKKNSSLHITATQTTDVGTYFCAGALPRAGSYQLT FGKGTKLSVIPN |
| 45 | atgctgtgttctctgctggctctgctgctgggcacottttttggcgtcagaagccagaccatccaccagtggcctgctacactggtgcagcctgttggaagccctctgagcctggaatgtaccgtggaaggcaccagcaatcccaacctgtactggtacagacaggccgctggaagaggactgcagctgctgttttacagcgtcggcatcggccagatcagcagcgaggttccacagaatctgagcgccagcagaccccaggacagacagtttatcctgagcagcaagaagctgctgctgagcgacagcggcttctacctgtgtgcttggagcctcggagccggctacaccgacacacagtattttggccctggcaccagactgaccgtgctg |
| 46 | MLCSLLALLLGTFFGVRSQTIHQWPATLVQPVGSPLSLECTVEGTSNPNLYWYRQAAGRG LQLLFYSVGIGQISSEVPQNLSASRPQDRQFILSSKKLLLSDSGFYLCAWSLGAGYTDTQY FGPGTRLTVL |
| 47 | atgctgctgatcacctccatgctggtgctgtggatgcagctgagccaagtgaacggccagcaagtgatgcagatccctcagta ccagcacgtgcaagaaggcgaggacttcaccacctactgcaacagcagcaccacactgagcaacatccagtggtacaag cageggcctggeggacaccctgtgtttctgatccagctggtcaagtccggcgaagtgaagaagcagaagcggctgaccttccagttcggegaggccaagaagaacagcagcctgcacatcaccgccacacagaccaccgatgtgggcacctacttttgtgctggcgccctgcctagagccggcagctatcaactgacattcggcaagggcaccaagctgagcgtgatccccaacatccagaatccggagcccgccgtataccagctgaaggaccctagaagccaggacagcaccctgtgcctgttcaccgacttcgacagccagatcaacgtgcccaagaccatggaaagcggcaccttcatcaccgacaagacagtgctggacatgaaggccatggacagcaagtccaacggcgcaatcgcctggtccaaccagaccagcttcacatgccaggacatcttcaaagagacaaacgccacataccccagcagcgacgtgccctgtgatgccaccctgacagagaagtccttcgagacagacatgaacctgaacttccagaatctgtccgtgatgggcctgagaatcctgctgctgaaggtggccggcttcaatctgctgatgaccctgcggctgtggtccagc |
| 48 | MLLITSMLVLWMQLSQVNGQQVMQIPQYQHVQEGEDFTTYCNSSTTLSNIQWYKQRPGG HPVFLIQLVKSGEVKKQKRLTFQFGEAKKNSSLHITATQTTDVGTYFCAGALPRAGSYQLT FGKGTKLSVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS |
| 49 | atgctgtgttctctgctggctctgctgctgggcaccttttttggcgtcagaagccagaccatccaccagtggcctgctacactggtg cagcctgttggaagccctctgagcctggaatgtaccgtggaaggcaccagcaatcccaacctgtactggtacagacaggccgctggaagaggactgcagctgctgttttacagcgtcggcatcggccagatcagcagcgaggttccacagaatctgagcgccagcagaccccaggacagacagtttatcctgagcagcaagaagctgctgctgagcgacagcggcttctacctgtgtgcttggagcctcggagccggctacaccgacacacagtattttggccctggcaccagactgaccgtgctggaagatctccggaacgtgaccccccctaaagtgaccctgttcgaacccagcaaggccgagatcgccaacaagcagaaagccaccctcgtgtgcctggccagaggcttcttccccgaccatgtggaactgtcttggtgggtcaacggcaaagaggtgcacagcggagtgtccaccgaccctcaggcctacaaagagagcaactacagctactgcctgagcagcagactgcgggtgtccgccaccttctggcacaacccccggaaccacttcagatgccaggtgcagtttcacggcctgagcgaagaggacaagtggcccgaaggctcccccaagcccgtgacccagaatatctctgccgaggcctggggcagagccgactgtggaattaccagcgccagctaccaccagggcgtgctgtctgccaccatcctgtacgagatcctgctgggcaaggccaccctgtacgccgtgctggtgtctggcctggtgctgatggccatggtcaagaagaagaacagc |
| 50 | MLCSLLALLLGTFFGVRSQTIHQWPATLVQPVGSPLSLECTVEGTSNPNLYWYRQAAGRG LQLLFYSVGIGQISSEVPQNLSASRPQDRQFILSSKKLLLSDSGFYLCAWSLGAGYTDTQY FGPGTRLTVLEDLRNVTPPKVTLFEPSKAEIANKQKATLVCLARGFFPDHVELSWWVNGK EVHSGVSTDPQAYKESNYSYCLSSRLRVSATFWHNPRNHFRCQVQFHGLSEEDKWPEG SPKPVTQNISAEAWGRADCGITSASYHQGVLSATILYElLLGKATLYAVLVSGLVLMAMVKK KNS |
| 51 | MERRRLWGSIQSRYISMSVWTSPRRLVELAGQSLLKDEALAIAALELLPRELFPPLFMAAF DGRHSQTLKAMVQAWPFTCLPLGVLMKGQHLHLETFKAVLDGLDVLLAQEVRPRRWKLQ VLDLRKNSHQDFWTVWSGNRASLYSFPEPEAAQPMTKKRKVDGLSTEAEQPFIPVEVLV DLFLKEGACDELFSYLIEKVKRKKNVLRLCCKKLKIFAMPMQDIKMILKMVQLDSIEDLEVTC TWKLPTLAKFSPYLGQMINLRRLLLSHIHASSYISPEKEEQYIAQFTSQFLSLQCLQALYVD SLFFLRGRLDQLLRHVMNPLETLSITNCRLSEGDVMHLSQSPSVSQLSVLSLSGVMLTDVS PEPLQALLERASATLQDLVFDECGITDDQLLALLPSLSHCSQLTTLSFYGNSISISALQSLLQ HLIGLSNLTHVLYPVPLESYEDIHGTLHLERLAYLHARLRELLCELGRPSMVWLSANPCPH CGDRTFYDPEPILCPCFMPN |
| 52 | YYSDSIFFL |
| 53 | LYVDTIGFL |
| 54 | DYVDSLYFC |
| 55 | LYYDHLGFL |
| 56 | DYVGTLFFL |

1. A T cell receptor (TCR) capable of binding to
a polypeptide comprising an amino acid sequence according to amino acid sequence LYVDSLFFL (SEQ ID NO: 2) wherein not more than 4 amino acids have been substituted, or
to a portion of said polypeptide, or
to the respective HLA-A bound form of said polypeptide or portion thereof,
wherein the TCR comprises:
(A) a CDR3
(Aa) of the TCR alpha chain comprising an amino acid sequence being at least 80% similar to SEQ ID NO: 12, and/or
(Ab) of the TCR beta chain comprising an amino acid sequence being at least 80% similar to SEQ ID NO: 14, or
(B) a CDR3
(Ba) of the TCR alpha chain comprising an amino acid sequence being at least 80% similar to SEQ ID NO: 40, and/or
(Bb) of the TCR beta chain comprising of an amino acid sequence being at least 80% similar to SEQ ID NO: 42.

2. The TCR according to claim 1,
wherein said TCR comprising a CDR3 according to (A) further comprises
(Aa1) a CDR1 of the TCR alpha chain comprising an amino acid sequence being at least 80% similar to the amino acid sequence of SEQ ID NO: 4, and/or a CDR2 of the TCR alpha chain comprising an amino acid sequence being at least 80% similar to the amino acid sequence of SEQ ID NO: 8, and/or
(Ab1) a CDR1 of the TCR beta chain comprising an amino acid sequence being at least 80% similar to the amino acid sequence of SEQ ID NO: 6, and/or a CDR2 of the TCR beta chain comprising an amino acid sequence being at least 80% similar to the amino acid sequence of SEQ ID NO: 10,
or wherein said TCR comprising a CDR3 according to (B) further comprises
(Ba1) a CDR1 of the TCR alpha chain comprising an amino acid sequence being at least 80% similar to the amino acid sequence of SEQ ID NO: 32, and/or a CDR2 of the TCR alpha chain comprising an amino acid sequence being at least 80% similar to the amino acid sequence of SEQ ID NO: 36, and/or
(Bb1) a CDR1 of the TCR beta chain comprising an amino acid sequence being at least 80% similar to the amino acid sequence of SEQ ID NO: 34, and/or a CDR2 of the TCR beta chain comprising an amino acid sequence being at least 80% similar to the amino acid sequence of SEQ ID NO: 38.

3. The TCR according to claims 1 or 2, wherein the HLA-A is an HLA-A*24, or HLA-A*02 encoded molecule.

4. The TCR according to any one of the preceding claims, wherein binding of said TCR to said polypeptide, or a portion thereof, or its HLA-A bound form, induces IFN-gamma secretion by cells comprising said TCR.

5. The TCR according to claim 4, wherein said induction of IFN-gamma secretion of cells comprising said TCR is at least 5-fold higher compared to control cells not comprising said TCR upon binding to a polypeptide comprising an amino acid sequence according to amino acid sequence LYVDSLFFL (SEQ ID NO: 2) wherein not more than 4 amino acids have been substituted, or to a portion of said polypeptide, or to the respective HLA-A bound form of said polypeptide or portion thereof.

6. The TCR according to any of the preceding claims,
wherein said TCR comprising a CDR3 according to (A) comprises
(Aa2) a TCR alpha chain variable region
comprising an amino acid sequence being at least 80% similar to SEQ ID NO: 16, and
comprising an amino acid sequence being at least 80% similar to positions 47 to 51 of SEQ ID NO: 16, and
comprising an amino acid sequence being at least 80% similar to positions 69 to 75 of SEQ ID NO: 16, and
comprising an amino acid sequence being at least 80% similar to positions 109 to 123 of SEQ ID NO: 16, and/or
(Ab2) a TCR beta chain variable region
comprising an amino acid sequence being at least 80% similar to SEQ ID NO: 18, and
comprising an amino acid sequence being at least 80% similar to positions 46 to 50 of SEQ ID NO: 18, and
comprising an amino acid sequence being at least 80% similar to positions 68 to 73 of SEQ ID NO: 18, and
comprising an amino acid sequence being at least 80% similar to positions 110 to 122 of SEQ ID NO: 18, or
wherein said TCR comprising a CDR3 according to (B) comprises
(Ba2) a TCR alpha chain variable region
comprising an amino acid sequence being at least 80% similar to SEQ ID NO: 44, and
comprising an amino acid sequence being at least 80% similar to positions 45 to 49 of SEQ ID NO: 44, and
comprising an amino acid sequence being at least 80% similar to positions 67 to 73 of SEQ ID NO: 44, and
comprising an amino acid sequence being at least 80% similar to positions 107 to 121 of SEQ ID NO: 44, and/or
(Bb2) a TCR beta chain variable region
comprising the amino acid sequence being at least 80% similar to SEQ ID NO: 46, and
comprising an amino acid sequence being at least 80% similar to positions 44 to 49 of SEQ ID NO: 46, and
comprising an amino acid sequence being at least 80% similar to positions 67 to 71 of SEQ ID NO: 46, and
comprising an amino acid sequence being at least 80% similar to positions 108 to 122 of SEQ ID NO: 46.

7. The TCR according to any of the preceding claims,
wherein said TCR comprising a CDR3 according to (A) comprises
(Aa3) a TCR alpha chain
comprising an amino acid sequence being at least 80% similar to SEQ ID NO: 20, and
comprising an amino acid sequence being at least 80% similar to positions 47 to 51 of SEQ ID NO: 20, and
comprising an amino acid sequence being at least 80% similar to positions 69 to 75 of SEQ ID NO: 20, and
comprising an amino acid sequence being at least 80% similar to positions 109 to 123 of SEQ ID NO: 20, and/or
(Ab3) a TCR beta chain
comprising an amino acid sequence being at least 80% similar to SEQ ID NO: 22, and
comprising an amino acid sequence being at least 80% similar to positions 46 to 50 of SEQ ID NO: 22, and
comprising an amino acid sequence being at least 80% similar to positions 68 to 73 of SEQ ID NO: 22, and
comprising an amino acid sequence being at least 80% similar to positions 110 to 122 of SEQ ID NO: 22, or
wherein said TCR comprising a CDR3 according to (B) comprises
(Ba3) a TCR alpha chain
comprising an amino acid sequence being at least 80% similar to SEQ ID NO: 48, and
comprising an amino acid sequence being at least 80% similar to positions 45 to 49 of SEQ ID NO: 48, and
comprising an amino acid sequence being at least 80% similar to positions 67 to 73 of SEQ ID NO: 48, and
comprising an amino acid sequence being at least 80% similar to positions 107 to 121 of SEQ ID NO: 48, and/or
(Bb3) a TCR beta chain
comprising an amino acid sequence being at least 80% similar to SEQ ID NO: 50, and
comprising an amino acid sequence being at least 80% similar to positions 44 to 49 of SEQ ID NO: 50, and
comprising an amino acid sequence being at least 80% similar to positions 67 to 71 of SEQ ID NO: 50, and
comprising an amino acid sequence being at least 80% similar to positions 108 to 122 of SEQ ID NO: 50.

8. The TCR according to any of the preceding claims, comprising
(A) at least one TCR alpha chain or subregion thereof according to (Aa), (Aa1), (Aa2) or (Aa3), and
at least one TCR beta chain or subregion thereof according to (Ab), (Ab1), (Ab2) or (Ab3),
covalently linked to each other to form a TCR heterodimer or multimer, or
(B) at least one TCR alpha chain or subregion thereof according to (Ba), (Ba1), (Ba2) or (Ba3), and
at least one TCR beta chain or subregion thereof according to (Bb), (Bb1), (Bb2) or (Bb3),
covalently linked to each other to form a TCR heterodimer or multimer.

9. A nucleic acid molecule encoding the TCR according to any one of the preceding claims.

10. The nucleic acid according to claim 9, comprising the nucleic acid sequence being at least 80% identical to the nucleic acid sequence of any one of SEQ ID NOs:, 3, 5, 7, 9, 11, 13, 15, 17, 19, or 21; or being at least 80% identical to the nucleic acid sequence of any one of SEQ ID NOs: 31, 33, 35, 37, 39, 41, 43, 45, 47, or 49.

11. A vector comprising the nucleic acid molecule according to claims 9 or 10.

12. A host cell comprising the TCR according to any one of claims 1 to 8, the nucleic acid molecule according to claim 9 or 10 or the vector according to claim 11.

13. A pharmaceutical or diagnostic composition comprising one or more of:
(i) the TCR according to any one of claims 1 to 8;
(ii) the nucleic acid molecule according to claim 9 or 10;
(iii) the vector according to claim 11; and/or
(iv) the host cell according to claim 12,
and, optionally, pharmaceutically excipient(s).

14. The TCR according to any one of claims 1 to 8, the nucleic acid molecule according to claim 9 or 10, the vector according to claim 11 and/or the host cell according to claim 12 for use as a medicament.

15. The TCR according to any one of claims 1 to 8, the nucleic acid molecule according to claim 9 or 10, the vector according to claim 11 and/or the host cell according to claim 12 for use in detection, diagnosis, prognosis, prevention and/or treatment of cancer.


SEQUENCE LISTING (56 sequences)

SEQ ID NO: 1 - DNA, 27 bp, Homo sapiens
ctctatgtggactctttatttttcctt

SEQ ID NO: 2 - protein, 9 aa, Homo sapiens
LYVDSLFFL

SEQ ID NO: 3 - DNA, 15 bp, Homo sapiens
aaggccctgtacagc

SEQ ID NO: 4 - protein, 5 aa, Homo sapiens
KALYS

SEQ ID NO: 5 - DNA, 15 bp, Homo sapiens
gagaaccatcggtac

SEQ ID NO: 6 - protein, 5 aa, Homo sapiens
ENHRY

SEQ ID NO: 7 - DNA, 21 bp, Homo sapiens
ctgctgaaaggcggcgagcag

SEQ ID NO: 8 - protein, 7 aa, Homo sapiens
LLKGGEQ

SEQ ID NO: 9 - DNA, 18 bp, Homo sapiens
agctacggcgtgaaggac

SEQ ID NO: 10 - protein, 6 aa, homo sapiens
SYGVKD

SEQ ID NO: 11 - DNA, 45 bp, Homo sapiens
tgcggcacagccaatagcggcggcagcaactacaagctgaccttc

SEQ ID NO: 12 - protein, 15 aa, Homo sapiens
CGTANSGGSNYKLTF

SEQ ID NO: 13 - DNA, 39 bp, Homo sapiens
tgcgccatcagcgactacgagggcaccgaggcctttttt

SEQ ID NO: 14 - protein, 13 aa, Homo sapiens
CAISDYEGTEAFF

SEQ ID NO: 15 - DNA, 402 bp, Homo sapiens
atggagacactgctgaaggtgctgtctggcacactgctgtggcagctgacctgggtccgatctcagcagcctgttcagtctcctcaggccgtgatcctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgcggcacagccaatagcggcggcagcaactacaagctgaccttcggcaagggcaccctgctgaccgtgaatcccaat

SEQ ID NO: 16 - protein, 134 aa, Homo sapiens
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTANSGGSNYKLTFGKGTLLTVNPN

SEQ ID NO: 17 - DNA, 393 bp, Homo sapiens
atgggcaccagactgttcttctacgtggccctgtgtctgctgtggacaggccatgtggatgccggaatcacacagagccccagacacaaagtgaccgagacaggcacccctgtgacactgagatgtcaccagaccgagaaccatcggtacatgtattggtacagacaggaccccggccacggcctgagactgatccactatagctacggcgtgaaggacaccgacaagggcgaagtgtctgacggctacagcgtgtccagaagcaagaccgaggacttcctgctgaccctggaaagcgccacaagcagccagaccagcgtgtacttctgcgccatcagcgactacgagggcaccgaggccttttttggccaaggcacaagactgaccgtggtg

SEQ ID NO: 18 - protein, 131 aa, Homo sapiens
MGTRLFFYVALCLLWTGHVDAGITQSPRHKVTETGTPVTLRCHQTENHRYMYWYRQDPGHGLRLIHYSYGVKDTDKGEVSDGYSVSRSKTEDFLLTLESATSSQTSVYFCAISDYEGTEAFFGQGTRLTVV

SEQ ID NO: 19 - DNA, 810 bp, Homo sapiens
atggagacactgctgaaggtgctgtctggcacactgctgtggcagctgacctgggtccgatctcagcagcctgttcagtctcctcaggccgtgatcctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgcggcacagccaatagcggcggcagcaactacaagctgaccttcggcaagggcaccctgctgaccgtgaatcccaatatccagaatccggagcccgccgtataccagctgaaggaccctagaagccaggacagcaccctgtgcctgttcaccgacttcgacagccagatcaacgtgcccaagaccatggaaagcggcaccttcatcaccgacaagacagtgctggacatgaaggccatggacagcaagtccaacggcgcaatcgcctggtccaaccagaccagcttcacatgccaggacatcttcaaagagacaaacgccacataccccagcagcgacgtgccctgtgatgccaccctgacagagaagtccttcgagacagacatgaacctgaacttccagaatctgtccgtgatgggcctgagaatcctgctgctgaaggtggccggcttcaatctgctgatgaccctgcggctgtggtccagc

SEQ ID NO: 20 - protein, 270 aa, Homo sapiens
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTANSGGSNYKLTFGKGTLLTVNPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 21 - DNA, 912 bp, Homo sapiens
atgggcaccagactgttcttctacgtggccctgtgtctgctgtggacaggccatgtggatgccggaatcacacagagccccagacacaaagtgaccgagacaggcacccctgtgacactgagatgtcaccagaccgagaaccatcggtacatgtattggtacagacaggaccccggccacggcctgagactgatccactatagctacggcgtgaaggacaccgacaagggcgaagtgtctgacggctacagcgtgtccagaagcaagaccgaggacttcctgctgaccctggaaagcgccacaagcagccagaccagcgtgtacttctgcgccatcagcgactacgagggcaccgaggccttttttggccaaggcacaagactgaccgtggtggaagatctccggaacgtgaccccccctaaagtgaccctgttcgaacccagcaaggccgagatcgccaacaagcagaaagccaccctcgtgtgcctggccagaggcttcttccccgaccatgtggaactgtcttggtgggtcaacggcaaagaggtgcacagcggagtgtccaccgaccctcaggcctacaaagagagcaactacagctactgcctgagcagcagactgcgggtgtccgccaccttctggcacaacccccggaaccacttcagatgccaggtgcagtttcacggcctgagcgaagaggacaagtggcccgaaggctcccccaagcccgtgacccagaatatctctgccgaggcctggggcagagccgactgtggaattaccagcgccagctaccaccagggcgtgctgtctgccaccatcctgtacgagatcctgctgggcaaggccaccctgtacgccgtgctggtgtctggcctggtgctgatggccatggtcaagaagaagaacagc

SEQ ID NO: 22 - protein, 304 aa, Homo sapiens
MGTRLFFYVALCLLWTGHVDAGITQSPRHKVTETGTPVTLRCHQTENHRYMYWYRQDPGHGLRLIHYSYGVKDTDKGEVSDGYSVSRSKTEDFLLTLESATSSQTSVYFCAISDYEGTEAFFGQGTRLTVVEDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 23 - DNA, 408 bp, Homo sapiens
atccagaatccggagcccgccgtataccagctgaaggaccctagaagccaggacagcaccctgtgcctgttcaccgacttcgacagccagatcaacgtgcccaagaccatggaaagcggcaccttcatcaccgacaagacagtgctggacatgaaggccatggacagcaagtccaacggcgcaatcgcctggtccaaccagaccagcttcacatgccaggacatcttcaaagagacaaacgccacataccccagcagcgacgtgccctgtgatgccaccctgacagagaagtccttcgagacagacatgaacctgaacttccagaatctgtccgtgatgggcctgagaatcctgctgctgaaggtggccggcttcaatctgctgatgaccctgcggctgtggtccagc

SEQ ID NO: 24 - protein, 136 aa, Homo sapiens
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 25 - DNA, 519 bp, Homo sapiens
gaagatctccggaacgtgaccccccctaaagtgaccctgttcgaacccagcaaggccgagatcgccaacaagcagaaagccaccctcgtgtgcctggccagaggcttcttccccgaccatgtggaactgtcttggtgggtcaacggcaaagaggtgcacagcggagtgtccaccgaccctcaggcctacaaagagagcaactacagctactgcctgagcagcagactgcgggtgtccgccaccttctggcacaacccccggaaccacttcagatgccaggtgcagtttcacggcctgagcgaagaggacaagtggcccgaaggctcccccaagcccgtgacccagaatatctctgccgaggcctggggcagagccgactgtggaattaccagcgccagctaccaccagggcgtgctgtctgccaccatcctgtacgagatcctgctgggcaaggccaccctgtacgccgtgctggtgtctggcctggtgctgatggccatggtcaagaagaagaacagc

SEQ ID NO: 26 - protein, 173 aa, Homo sapiens
EDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 27 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 28 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 29 - protein, 140 aa, Mus musculus
IQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 30 - protein, 177 aa, Homo sapiens
EDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 31 - DNA, 15 bp, Homo sapiens
accacactgagcaac

SEQ ID NO: 32 - protein, 5 aa, Homo sapiens
TTLSN

SEQ ID NO: 33 - DNA, 18 bp, Homo sapiens
ggcaccagcaatcccaac

SEQ ID NO: 34 - protein, 6 aa, Homo sapiens
GTSNPN

SEQ ID NO: 35 - DNA, 21 bp, Homo sapiens
ctggtcaagtccggcgaagtg

SEQ ID NO: 36 - protein, 7 aa, Homo sapiens
LVKSGEV

SEQ ID NO: 37 - DNA, 15 bp, Homo sapiens
agcgtcggcatcggc

SEQ ID NO: 38 - protein, 5 aa, Homo sapiens
SVGIG

SEQ ID NO: 39 - DNA, 45 bp, Homo sapiens
tgtgctggcgccctgcctagagccggcagctatcaactgacattc

SEQ ID NO: 40 - protein, 15 aa, Homo sapiens
CAGALPRAGSYQLTF

SEQ ID NO: 41 - DNA, 45 bp, Homo sapiens
tgtgcttggagcctcggagccggctacaccgacacacagtatttt

SEQ ID NO: 42 - protein, 15 aa, Homo sapiens
CAWSLGAGYTDTQYF

SEQ ID NO: 43 - DNA, 396 bp, Homo sapiens
atgctgctgatcacctccatgctggtgctgtggatgcagctgagccaagtgaacggccagcaagtgatgcagatccctcagtaccagcacgtgcaagaaggcgaggacttcaccacctactgcaacagcagcaccacactgagcaacatccagtggtacaagcagcggcctggcggacaccctgtgtttctgatccagctggtcaagtccggcgaagtgaagaagcagaagcggctgaccttccagttcggcgaggccaagaagaacagcagcctgcacatcaccgccacacagaccaccgatgtgggcacctacttttgtgctggcgccctgcctagagccggcagctatcaactgacattcggcaagggcaccaagctgagcgtgatccccaac

SEQ ID NO: 44 - protein, 132 aa, Homo sapiens
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGALPRAGSYQLTFGKGTKLSVIPN

SEQ ID NO: 45 - DNA, 393 bp, Homo sapiens
atgctgtgttctctgctggctctgctgctgggcaccttttttggcgtcagaagccagaccatccaccagtggcctgctacactggtgcagcctgttggaagccctctgagcctggaatgtaccgtggaaggcaccagcaatcccaacctgtactggtacagacaggccgctggaagaggactgcagctgctgttttacagcgtcggcatcggccagatcagcagcgaggttccacagaatctgagcgccagcagaccccaggacagacagtttatcctgagcagcaagaagctgctgctgagcgacagcggcttctacctgtgtgcttggagcctcggagccggctacaccgacacacagtattttggccctggcaccagactgaccgtgctg

SEQ ID NO: 46 - protein, 131 aa, Homo sapiens
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSLGAGYTDTQYFGPGTRLTVL

SEQ ID NO: 47 - DNA, 804 bp, Homo sapiens
atgctgctgatcacctccatgctggtgctgtggatgcagctgagccaagtgaacggccagcaagtgatgcagatccctcagtaccagcacgtgcaagaaggcgaggacttcaccacctactgcaacagcagcaccacactgagcaacatccagtggtacaagcagcggcctggcggacaccctgtgtttctgatccagctggtcaagtccggcgaagtgaagaagcagaagcggctgaccttccagttcggcgaggccaagaagaacagcagcctgcacatcaccgccacacagaccaccgatgtgggcacctacttttgtgctggcgccctgcctagagccggcagctatcaactgacattcggcaagggcaccaagctgagcgtgatccccaacatccagaatccggagcccgccgtataccagctgaaggaccctagaagccaggacagcaccctgtgcctgttcaccgacttcgacagccagatcaacgtgcccaagaccatggaaagcggcaccttcatcaccgacaagacagtgctggacatgaaggccatggacagcaagtccaacggcgcaatcgcctggtccaaccagaccagcttcacatgccaggacatcttcaaagagacaaacgccacataccccagcagcgacgtgccctgtgatgccaccctgacagagaagtccttcgagacagacatgaacctgaacttccagaatctgtccgtgatgggcctgagaatcctgctgctgaaggtggccggcttcaatctgctgatgaccctgcggctgtggtccagc

SEQ ID NO: 48 - protein, 268 aa, Homo sapiens
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGALPRAGSYQLTFGKGTKLSVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 49 - DNA, 912 bp, Homo sapiens
atgctgtgttctctgctggctctgctgctgggcaccttttttggcgtcagaagccagaccatccaccagtggcctgctacactggtgcagcctgttggaagccctctgagcctggaatgtaccgtggaaggcaccagcaatcccaacctgtactggtacagacaggccgctggaagaggactgcagctgctgttttacagcgtcggcatcggccagatcagcagcgaggttccacagaatctgagcgccagcagaccccaggacagacagtttatcctgagcagcaagaagctgctgctgagcgacagcggcttctacctgtgtgcttggagcctcggagccggctacaccgacacacagtattttggccctggcaccagactgaccgtgctggaagatctccggaacgtgaccccccctaaagtgaccctgttcgaacccagcaaggccgagatcgccaacaagcagaaagccaccctcgtgtgcctggccagaggcttcttccccgaccatgtggaactgtcttggtgggtcaacggcaaagaggtgcacagcggagtgtccaccgaccctcaggcctacaaagagagcaactacagctactgcctgagcagcagactgcgggtgtccgccaccttctggcacaacccccggaaccacttcagatgccaggtgcagtttcacggcctgagcgaagaggacaagtggcccgaaggctcccccaagcccgtgacccagaatatctctgccgaggcctggggcagagccgactgtggaattaccagcgccagctaccaccagggcgtgctgtctgccaccatcctgtacgagatcctgctgggcaaggccaccctgtacgccgtgctggtgtctggcctggtgctgatggccatggtcaagaagaagaacagc

SEQ ID NO: 50 - protein, 304 aa, Homo sapiens
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSLGAGYTDTQYFGPGTRLTVLEDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 51 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 52 - protein, 9 aa, artificial
  mutated peptide
YYSDSIFFL

SEQ ID NO: 53 - protein, 9 aa, artificial
  mutated peptide
LYVDTIGFL

SEQ ID NO: 54 - protein, 9 aa, artificial
  mutated peptide
DYVDSLYFC

SEQ ID NO: 55 - protein, 9 aa, artificial
  mutated peptide
LYYDHLGFL

SEQ ID NO: 56 - protein, 9 aa, artificial
  mutated peptide
DYVGTLFFL